(19) United States
(12) Patent Application Publication
(10) Pub. No.: US 2011/0093032 A1
(43) Pub. Date: Apr. 21, 2011

(54) Systems and methods for maintaining airway patency

(75) Inventors: Joseph W. Boggs, II, Carrboro, NC (US); Paul Byong Suk Yoo, Durham, NC (US)

(73) Assignee: NDI Medical, LLC

(21) Appl. No.: 12/806,070

(22) Filed: Aug. 5, 2010

Related U.S. Application Data
(60) Provisional application No. 61/273,534, filed on Aug. 5, 2009.

(51) Int. Cl. A61N 1/36

(52) U.S. Cl. 607/42

(57) ABSTRACT

Systems and methods according to the present invention use an electrical pulse generator system, which may be external to or implanted in an animal body, to provide therapeutically effective electrical stimulation to maintain or improve airway patency, such as to treat sleep apnea by the stimulation of target nerve(s) or their branches using one or more leads and one or more electrodes implanted in, on, around, or near the target nerve(s). Examples of a target nerves to be stimulated to maintain or improve upper airway patency, preferably through upper airway muscle reflex activation, are the internal branch of the superior laryngeal nerve (iSLN), the glossopharyngeal nerve, and/or the trigeminal nerve, and/or any of the trunks, branches, or divisions of such nerves.

### RELATED APPLICATIONS

This application claims the benefit of U.S. Provisional Patent Application Ser. No. 61/273,534, filed Aug. 5, 2009, and entitled “Systems and Methods Provide Stimulation of Nerves and/or Muscles for the Treatment of Sleep Apnea,” which is incorporated herein by reference in its entirety.

### FIELD OF THE INVENTION

This invention relates generally to systems and methods for providing electrical stimulation to tissue, more specifically to systems and methods for maintaining upper airway patency, which may include electrical stimulation of nerves and/or muscles to treat sleep apnea.

### BACKGROUND OF THE INVENTION

Sleep-Disordered breathing (SDB) generally describes a group of disorders that are characterized by abnormalities of respiratory pattern (usually pauses in breathing) or the quantity of ventilation during sleep. Obstructive sleep apnea (OSA), the most common such disorder, is characterized by the repetitive collapse or partial collapse of the pharyngeal airway during sleep and associated repeated arousal, full or partial, required to resume ventilation. Sleep is thus disrupted, sometimes without notice, yielding waking somnolence and diminished neurocognitive performance. The recurrent sleep arousal in association with intermittent hypoxia and hypercapnia has been implicated in the occurrence of adverse cardiovascular outcomes. In addition, there is evolving evidence that SDB may contribute to insulin resistance and other components of the metabolic syndrome. Despite considerable progress, most patients remain undiagnosed. Obstructive sleep apnea (OSA) is characterized by repeated nocturnal narrowing (hypopnea) and obstruction or collapse (apnea) of the upper airway (UA) that results in nocturnal arterial oxygen desaturation, increased arterial pressure, elevated heart rate, micro-arousals, and sleep fragmentation. Due in part to these repeated acute physiological perturbations throughout each night, the cumulative long-term effects of OSA have been linked to hypertension, acute myocardial infarction, chronic heart failure, stroke, excessive daytime sleepiness, and increased incidence of automobile accidents. The primary causes of OSA include small airway caliber (e.g., due to obesity) and inadequate activation of UA dilator muscles (e.g., tongue and palatal muscles). In the United States alone, it has been estimated that 12 million people (4% of adult population), and up to 24% of individuals over 65 years of age, are affected by OSA.

Although first recognized clinically in 1965, only recently has OSA been recognized as a significant medical condition that affects the quality of life and even cognitive function in people that suffer from OSA. The socio-economic burden of this syndrome is also remarkable. According to one recent study, which examined the impact of OSA on automobile accidents in the United States, the estimated total cost in the year 2000 alone exceeded 15.9 billion US dollars, resulting in the loss of 1400 lives, and involving approximately 800,000 persons in motor-vehicle collisions. Another study conducted in Australia estimated the total economic burden of sleep disorders, primarily including OSA, at 7.5 billion US dollars. If extrapolated to the current US adult population, the estimated cost for the US would exceed that of both asthma and chronic obstructive pulmonary disease, and almost approach that of diabetes. With the current aging US population, the economic burden of OSA is further underscored by the projected increase in the utilization rate of the healthcare system, which has been shown to increase proportionately with both the diagnosis and severity of OSA symptoms.

Prior methods of OSA treatment, which include continuous positive airway pressure (CPAP) therapy and pharyngeal surgery, are generally unsatisfactory because of low patient compliance and highly unpredictable therapeutic outcomes, respectively. It has also previously been shown that airway patency may also be improved by electrical stimulation of the hypoglossal (HG) nerve, but methods presently under investigation fail to provide consistent therapeutic efficacy. Although direct activation of the genioglossus muscle (i.e., tongue protrudor) might improve OSA symptoms, such activation often fails to reduce the number of apneic events below the threshold for diagnosing OSA.

Clinical outcomes of current therapies are difficult to predict. The gold standard for treating OSA involves the application during sleep of positive airway pressure (PAP)—of which CPAP is the most prevalent. This is commonly referred to as CPAP therapy (commercially available by Philips Respironics, Inc and Resmed Inc.) and consists of a pressure pump connected to a nasal mask worn by the patient. Other types of PAP may be used, including VPAP or BiPAP (Variable/Bilevel Positive Airway Pressure). They are similar to CPAP but provide two levels of pressure: one (higher) Inspiratory Positive Airway Pressure (IPAP) and one (lower) Expiratory Positive Airway Pressure (EPAP) for easier exhalation. By raising the intraluminal pressure (Pᵢₙ) along the UA, the system acts as a pneumatic splint that effectively lowers the airway pressure (i.e., P_{crit}) at which airway instability (i.e., flow limitation) or collapse occurs, thus reducing the number of apneic events during sleep. The frequency of apneic events per hour of sleep is termed the apnea hypopnea index (AHI) and is used clinically to diagnose and measure the severity of OSA (clinical threshold is AHI=5). CPAP effectively resolves airway obstructions (AHI<5), but patients consider the mask cumbersome or uncomfortable, many indicate other side effects (e.g., nasal drying, rhinitis, ear pain, and conjunctivitis), and the noise generated by the machine frequently disturbs the bed partner. Clinical studies have shown compliance rates of only approximately 40%, where over half of the individuals prescribed with a CPAP machine discontinue use within one year. Still other studies appear to demonstrate that a small subset of OSA patients may develop central sleep apnea with CPAP therapy.

More conventional approaches to treat OSA may be described as an attempt to correct the anatomical factors that predispose individuals to UA obstruction. These include uvulopalatopharyngoplasty (UPPP or UP3), radio frequency ablation of the tongue, mandibular or tongue advancement surgery, and oral appliances. Although therapeutic effectiveness is frequently reported, significant limitations are indicated by the overall consistency of the outcomes and the difficultly in predicting both the immediate and long-term benefits for a given procedure. Oral appliances are effective clinically in only about 50% of patients despite relatively high compliance rates (77%) after the first year. Similarly, there is no preoperative variable (e.g., severity of OSA or anatomical characteristic) that can reliably predict successful outcome of pharyngeal surgery. Additional surgeries are often performed to improve the outcome. In extreme cases, a tracheostomy may offer a highly effective solution that bypasses completely the collapsible pharynx. However, this procedure is poorly tolerated by patients and rarely used due to stomal infections, chronic cough, episodes of dyspnea, and aesthetic reasons.

The need for an alternative to CPAP therapy and surgery has advanced the commercial development of HG nerve stimulation. The intended therapeutic effect of HG nerve stimulation is to prevent obstruction within the velopharynx, the most common site of airway closure. This treatment is achieved by direct electrical activation of the tongue protrudor (genioglossus) muscle, which in turn inhibits posterior prolapse of the tongue against the soft palate.

Although early clinical data of HG nerve therapy demonstrated significant improvements from severe to moderate OSA, the average post-therapy AHI (22.6±12.1) remained above the clinical threshold of AHI=5, while also exhibiting significant inter-patient variability. According to some recent studies, airway patency may also depend on concomitant activation of additional muscles that stiffen the airway and improve UA stability (i.e., lower P_{crit}): tongue retractor (styloglossus and hyoglossus) and palatal (tensor veli palatini) muscles. Moreover, dynamic changes in both the site of airway obstruction and UA muscle tone during different stages of sleep also likely contribute to the pathogenesis of OSA. HG nerve therapy fails to address all of these factors.

It is time that systems and methods for electrical stimulation of nerves and/or muscles address not only specific objectives relating to the treatment of sleep apnea, but also address the quality of life of the individual requiring the treatment and the bed partner thereof.

### SUMMARY OF THE INVENTION

It has been discovered that the incidence of OSA involves more than the selective loss of GG muscle activity; hence the limited therapeutic efficacy achieved by HG nerve stimulation, as mentioned above.

Electrical stimulation of target nerves, including but not limited to the internal branch of the superior laryngeal nerve (iSLN), and/or the glossopharyngeal nerve, and/or the trigeminal nerve, and/or their roots and/or branches, in combination, or individually, has been discovered to improve airway patency. It is to be appreciated that when describing the systems and methods and referencing the iSLN, any of the target nerves and/or their branches take the place of, or may be used in combination with the iSLN.

The systems and methods of the present invention are based on the critical role of the afferent iSLN (and the other potential target nerves) mediated UAD reflex in maintaining airway patency during sleep. In animal studies, bilateral transection of the iSLN or topical anesthesia applied to the airway lumen results in a significant reduction of reflex airway dilator muscle activity. A similar decrease in reflex dilator muscle activity is also observed in airway-anesthetized humans, which during sleep promotes the emergence of apneic symptoms in otherwise healthy individuals. Electrical stimulation of the iSLN may augment the UAD reflex and thereby evoke a significant increase in airway patency.

Electrical activation of the internal branch of the superior laryngeal nerve (iSLN) offers a simple alternative approach to treating OSA by augmenting the upper airway dilator (UAD) reflex via afferent fibers that innervate both functional and non-functional (e.g., inflammatory damage) airway receptors. The rationale for this method is based on pathological changes in airway mechanoreceptor function in OSA patients that result from chronic inflammatory-type damage to mucosal receptors from repeated snoring, and also by sub-mucosal accumulation of adipose tissue. This loss in normal airway sensation is thought to play a critical role in rendering the airway (e.g. upper airway) vulnerable to flow-limitation and eventual collapse during sleep. The advantages of this afferent approach to treating OSA include reflex activation of multiple airway dilating muscles (genioglossus and tensor veli palatini) and electrical stimulation of sensory fibers innervating airway receptors with limited sensory function. The minimally-invasive percutaneous approach to accessing the iSLN may provide an additional tool with which clinical professionals (e.g., an ear, nose and throat (E.N.T.) specialist or otolaryngologist) can use to evaluate and predict therapeutic efficacy.

Other features and advantages of embodiments of the present invention are set forth in the following specification and attached drawings.

### BRIEF DESCRIPTION OF THE DRAWINGS

FIG. 1 is a schematic representation of an embodiment of a system according to the present invention.

FIG. 2A is a right elevation view of a partial dissection of a human larynx region, including an implanted electrode in a first orientation according to the system of FIG. 1.

FIG. 2B is a right elevation view of a partial dissection of a human larynx region, including an implanted electrode in a second orientation according to the system of FIG. 1.

FIG. 3 is a medial cross-section of a human

FIG. 4 is an anatomical view depicting the efferent path from the hypoglossal motor nucleus to various airway dilator muscles.

FIGS. 5A and 5B diagrammatically depict the relationship between afferent target nerves and efferent activation of airway dilator muscles.

FIG. 6 depicts a Starling resistor model.

FIG. 7 is a table comparing systems and methods according to the present invention with prior treatment mechanisms for obstructive sleep apnea.

FIG. 8 is a flow chart depicting an embodiment of a method according to the present invention.

FIG. 9 is a graphical comparison of nasal pressure versus airway velocity between a healthy individual and an individual that may be experiencing sleep apnea.

FIG. 10 is a graph of experimental data showing superimposed, measured EMG activity of the genioglossus and tensor palatini muscles in response to a single electrical stimulation of a target nerve.

FIG. 11 is a graph of experimental data showing measured EMG activity of the genioglossus and tensor palatini muscles in response to continuous electrical stimulation of a target nerve.

FIG. 12 is a graph of experimental data showing bilateral response of genioglossus muscle evoked by unilateral stimulation of a target nerve.

FIG. 13 is a graph of experimental data showing amplitude- and frequency-dependent activation of reflex genioglossus muscle activity.

FIG. 14 is a graph of experimental data showing frequency-dependent activation of reflex activity in genioglossus and tensor veli palatini muscles.

FIG. 15 is a graph of experimental data showing recruitment of reflex genioglossus muscle in response to target nerve stimulation.

FIG. 16 is a graph of experimental data showing a decrease in P_{crit}, indicating an improved airway patency with an increase in amplitude of electrical stimulation applied to the target nerve.

FIG. 17 is a graph of experimental data showing a decrease in P_{crit}, indicating an improved airway patency with an increase in amplitude of electrical stimulation applied to the target nerve.

FIG. 18 is a graph of experimental data showing a decrease in P_{crit}, indicating an improved airway patency associated with various frequencies of electrical stimulation applied to the target nerve.

FIG. 19 is a graph of experimental data showing a linear correlation between a decrease in P_{crit}, indicating an improved airway patency, and reflex genioglossus muscle activity evoked by electrical stimulation applied to a target nerve.

FIG. 20 is a perspective view of a step of electrode insertion according to an embodiment of a method according to the present invention.

FIGS. 21-26 are anatomical reference figures.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

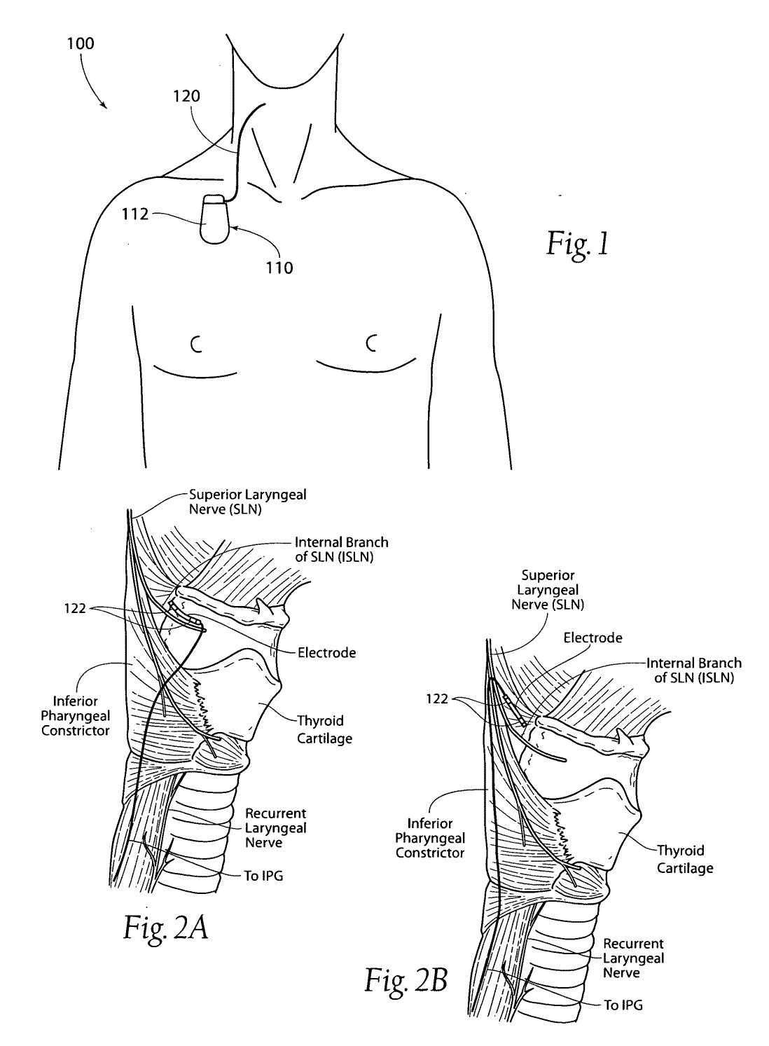

Referring now to the figures, FIGS. 1 and 2 schematically represent an embodiment of a system 100 for maintaining or promoting airway patency which may be used in the treatment of sleep-disordered breathing, such as sleep apnea, including obstructive sleep apnea (OSA). An electrical pulse generator 110, such as a surgically implanted pulse generator (IPG) 112 may be connected to or integrated with a uni- or multi-polar electrode 120 placed in the neck, head or chest of an animal, within a therapeutically effective range of one or more target nerves, such as the glossopharyngeal nerve, the trigeminal nerve, and/or the internal branch of the superior laryngeal nerve (iSLN), and/or any of the trunks, branches, or divisions of such nerves. Selective stimulation of target nerves may be performed through precise electrode placement or directional electrode design so as to stimulate only a desired single or plurality of target nerves without activation of untargeted nerves. Where a plurality of target nerves is stimulated, each nerve may be stimulated synchronously with the other(s), a synchronously from the other(s), or in regular or irregular sequence with the other(s). These target nerves provide sensory innervation to the airway (e.g. the upper airway) and also mediate an airway dilator (AD) reflex, namely the upper AD (UAD) reflex. The electrode may have one or more conductive surfaces or contacts 122. If more than one conductive surface 122 is provided, the surfaces 122 may be electrically coupled or electrically isolated on the electrode to enable independent activation and programmable operation of each surface 122 as an anode or cathode.

Embodiments of systems and methods according to the present invention may be used in maintaining, restoring and/or improving airway patency based on a reflex activation of at least one airway dilating muscle (e.g., both tongue and palatal muscles), which may include pharyngeal and/or laryngeal musculature. Muscle activation may be achieved by electrically evoking an AD reflex, or activating AD muscles, which is mediated by afferent target nerves. The target nerve(s) may form a part of the glossopharyngeal nerve, the trigeminal nerve, and/or the internal branch of the superior laryngeal nerve (iSLN), for example. A preferred AD reflex is the upper airway dilator (UAD) reflex, including the activation of UAD muscles, again which may include muscles located in or extending through the pharynx and/or larynx.

Electrical stimulation of a target nerve may activate both the tongue and palatal muscles, and thus decrease the propensity of the UA to obstruct during application of negative fluid pressure pulses, such as during inspiration. This may be confirmed by measuring physiological parameters, such as the electrical activity of muscles (electromyography (EMG)), electroneurogram (ENG), electrooculogram (EOG), electroencephalogram (EEG), electrocardiogram (ECG), blood oxygen levels, diaphragm movement, snoring, frequency or duration of apneic events, frequency or duration hypopneic events, airway flow (e.g. airway flow rates or velocity), and/or airway pressures (e.g. critical airway pressure (P_{crit}) at which flow limitation occurs) with and without electrical stimulation of the target nerve. The improvement in any physiological parameter(s) provided by target afferent stimulation may be compared to the improvement in any physiological parameter(s) evoked by other therapies, such as hypoglossal nerve stimulation, or in the absence of other therapies.

The UAD reflex may persist during and/or after sustained electrical stimulation of a target nerve. This may be confirmed by comparing time-dependent changes in the periodically measured physiological parameters in response to continuous vs. intermittent (i.e., regular, irregular, and/or varied duty cycle) electrical stimulation of the target nerve, which may occur over a period of seconds, minutes, hours, or days. Electrical stimulation according to the present invention may be performed according to a regular, periodic schedule, at irregular bursts of activation, or at discrete periods of activation initiated by an external or internal controller.

In addition to direct nerve stimulation, other types of stimulation that activate a target nerve may achieve, improve, or maintain airway patency (which may be measured with physiological parameters, such as those named above, including airway pressures (e.g. P_{crit})) that is comparable to that produced by direct nerve stimulation. Other types of electrical stimulation may include stimulation delivered by electrode contact(s) not in direct contact with the nerve but that are near or some distance away from the nerve. The achievement of, improvement in, or maintenance of airway patency may be confirmed by measuring physiological parameters in response to stimulating the target nerve with one or more percutaneously placed electrodes or leads, which may be placed unilaterally or bilaterally. The percutaneously placed leads may exit the body, thus remaining in a percutaneous arrangement, or they may be completely implanted within the body, having been introduced percutaneously. Percutaneous placement or introduction provides many benefits. For instance, such introduction minimizes procedure time, tissue damage, and recovery time. Further, it makes the method easier to perform, thereby lessening required clinician proficiency training.

As noted above, OSA imposes significant societal and economic costs. To understand OSA and the need for more effective therapy, it is advantageous to consider the anatomy and the pathophysiology involved.

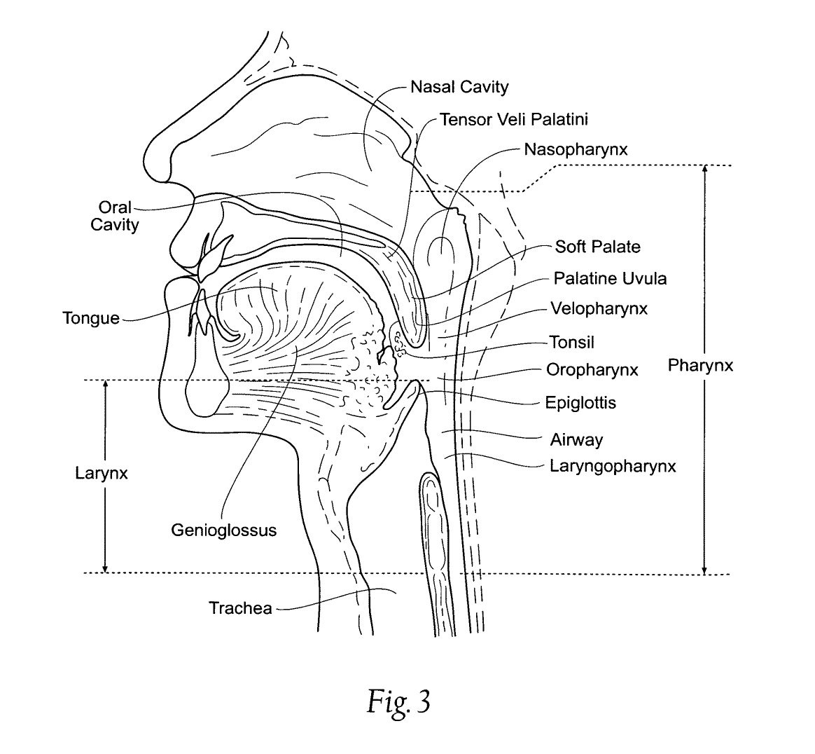

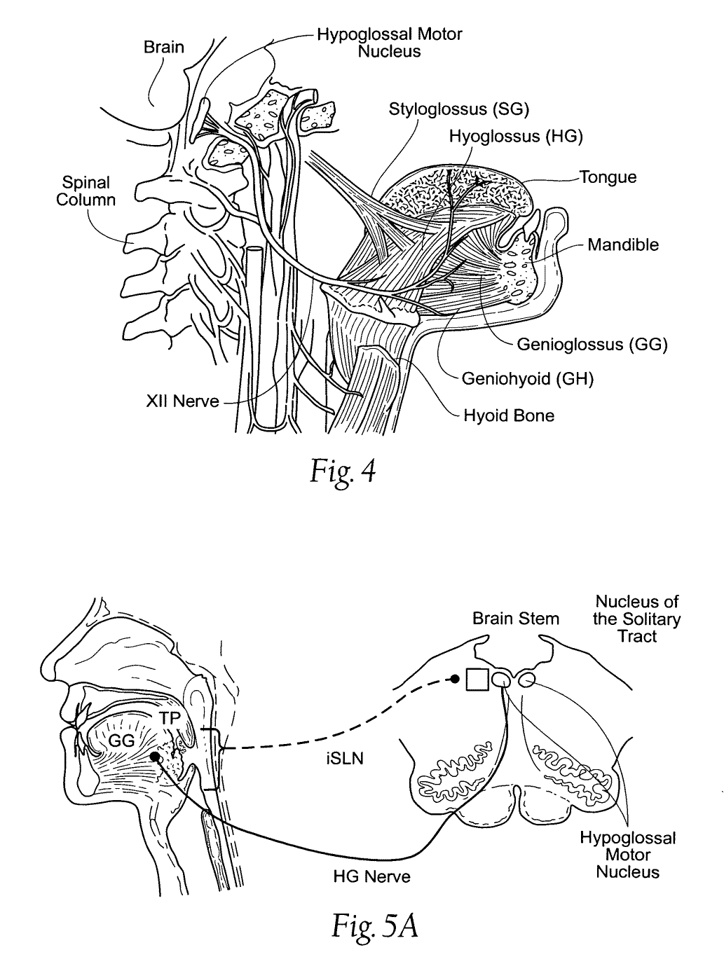

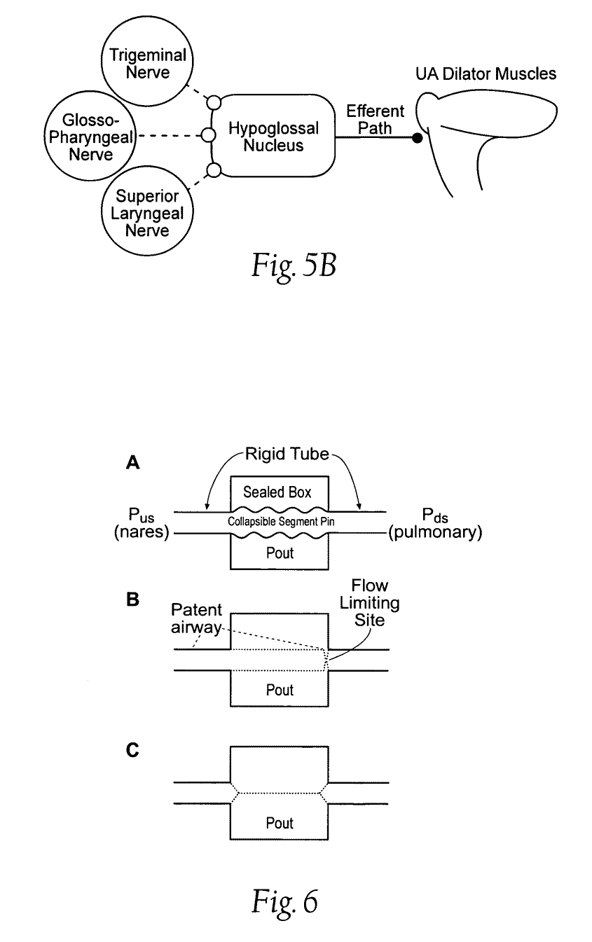

The human upper airway (UA) plays a critical role in maintaining proper respiratory function. This is achieved by the coordinated activation of specific muscle groups that anatomically define this segment of the airway. As shown in FIGS. 3 and 4, the UA consists of four regions: a nasopharynx, a velopahrynx, an oropharynx, and a laryngo-pharynx. The four UA regions may be differentiated physically by several key muscle groups: palatal (tensor veli palatini), tongue protrudor (genioglossus, geniohyoid), tongue retractor (styloglossus, hyoglossus) and pharyngeal constrictor muscles. Efferent innervation of these muscles is achieved by nerve branches derived from the trigeminal, hypoglossal and vagus nerves. In healthy individuals, upper airway patency is achieved by reflex activation of airway dilator muscles (genioglossal (GG) and tensor veli palatini (TP), FIG. 3), which respond to negative inspiratory pressure. This mechanism is referred to as the upper airway dilator (UAD) reflex or negative pressure reflex, and is mediated by sensory nerves (e.g., target nerves, such as the internal branch of the superior laryngeal nerve (iSLN), the glossopharyngeal nerve, and the trigeminal nerve) that innervate the airway lumen. These afferent fibers project into the central nervous system (e.g. onto brainstem nuclei such as the hypoglossal nucleus), which in turn elicits sustained contraction of UA dilator muscles (GG and TP muscles). The hypoglossal motor nucleus provides efferent input, via the hypoglossal (XII) nerve, to the tongue protrudor muscle (GG), tongue retractors (hyoglossus and styloglossus) and geniohyoid muscle. In healthy individuals, this reflex generates sufficient efferent output to the UAD muscles to help maintain airway patency, even during sleep. FIGS. 5A and 5B schematically depict the upper airway dilator (UAD) reflex. Mechanoreceptors along the upper airway (UA) lumen transduce afferent neural activity to the central nervous system (e.g. the nucleus of the solitary tract), via one or more target nerves. The central nervous system (e.g. the nucleus), in turn sends projections (e.g. to brain stem nuclei such as the hypoglossal nucleus) that excite efferent axons innervating target muscles, such as the airway dilators (e.g. genioglossus (GG) and tensor veli palatini (TP) muscles).

An insufficient activation of the upper airway dilator (UAD) reflex can lead to obstructive sleep apnea (OSA). The UAD reflex plays an important role in maintaining airway patency during sleep, but is compromised in persons with OSA. In awake OSA patients, the output of the UAD reflex is significantly increased as part of a compensatory mechanism aimed at counteracting both the hypopneic (e.g., para-pharyngeal fat pads) and apneic (e.g., negative inspiratory pressure) factors that lead to OSA. In contrast, the efferent output of this reflex is diminished in OSA patients during sleep, thus leading to further narrowing (i.e., flow limitation) and eventual collapse of the upper airway. Application of topical anesthesia along the upper airway lumen in healthy individuals produces symptoms of OSA, which includes an increase in resistance to airflow, the occurrence of flow limitation, and emergence of apneic events. Interestingly, the same experimental procedure applied to OSA patients is not known to produce any noticeable changes nor does it significantly affect the apnea hypopnea index (AHI, severity of OSA). This lack of response to UA anesthesia may have been the result of an elevated mechanical threshold of the airway receptors responsible for activating the UAD reflex during sleep. This is supported by the diminished sensitivity along the upper airway that may be associated with degeneration of mucosal mechanoreceptors by inflammation-type mechanisms. This loss in mechanoreceptor function can result from repetitive mechanical vibration of the UA during snoring and is thought to exacerbate the pathological effects of adipose tissue accumulation along the parapharyngeal submucosa, commonly observed with obesity in OSA. Thus, the cumulative loss of ‘respiratory sensation’ in OSA patients contributes to the physiological factors conducive to apneic events.

Activation of both the genioglossus (GG) and tensor veli palatini (TP) muscles are important for upper airway patency. The segment of the upper airway posterior to the soft palate (see velopharynx in FIG. 3) represents the most common site of obstruction in OSA patients. This is attributed primarily to the paradoxical loss of genioglossus (GG) muscle activity during sleep, which in turn causes prolapse of the tongue and compression of the soft palate onto the posterior pharyngeal wall. Activation of the GG muscle alone, however, is often not sufficient for maintaining velopharyngeal patency or complete elimination of apneic episodes in OSA patients. The palatal muscles (e.g., tensor veli palatini (TP)) also play a critical role in airway patency by increasing the overall stiffness of the soft palate and preventing adhesion between the velopharyngeal walls.

Thus, the importance of the UAD reflex in maintaining UA patency is further underscored by the activation of both the GG and TP muscles in response to negative airway pressures during sleep. The GG muscle may exhibit a phasic pattern of activity in synchrony with negative inspiratory airway pressure swings; whereas the TP muscle may display a tonic response to airway inputs. The pathogenic role of a diminished UAD reflex in OSA patients is also supported by the increase in apneic events following topical anesthesia of the pharynx in healthy individuals, as mentioned above. It has been suggested that the occurrence of such reflexes is likely independent of central mechanisms modulating UA dilator muscles.

Some of the mechanical effects of UAD muscle activity can be described by a Starling resistor model as shown in FIG. 6, where the difference between the intraluminal pressure within the collapsible segment (Pᵢₙ) and the pressure of the surrounding tissue (Pₒᵤₜ) determines the rate of inspiratory airflow and the collapsibility of the UA. In OSA patients, this collapsible segment corresponds primarily to the velopharynx. As shown in FIG. 6.A, if Pᵢₙ>Pₒᵤₜ, normal inspiratory airflow is determined by the pressure difference between upstream pressure (Pᵤₛ, atmosphere) and the downstream pressure (P_{ds}, pulmonary). If Pᵢₙ approximates Pₒᵤₜ (FIG. 6.B), the airway begins to narrow and flow limitation occurs, where further increase in P_{ds} fails to increase the overall rate of airflow. The Pᵢₙ at which flow limitation occurs is referred to as the critical pressure (P_{crit}), below which the UA collapses completely (FIG. 6.C). Given these properties of the UA, sufficient activation of both the GG and TP muscle is vital for maintaining UA patency.

As described above, clinical outcome of presently available therapies are difficult to predict. However, electrical stimulation of target nerves, such as the internal branch of the superior laryngeal nerve (iSLN), trigeminal nerve, and/or glossopharyngeal nerve, evokes reflex activation of upper airway (UA) dilator muscles. Embodiments of systems and methods according to the present invention may be used to stimulate electrically target nerves, such as the glossopharyngeal nerve, the trigeminal nerve, and/or the internal branch of the superior laryngeal nerve (iSLN), and/or any of the trunks, branches, or divisions of such nerves, to maintain or restore airway patency in persons with obstructive sleep apnea (OSA). One or more of the target nerves serves as a sensory pathway (e.g. a pharyngeal sensory pathway) that can be used as a neural substrate for treating OSA, as depicted by a dotted line with reference to the iSLN in FIG. 5. The utility of this afferent nerve has been confirmed by robust reflex activation of both the genioglossus (GG) and tensor veli palatini (TP) muscles in response to electrical stimulation of the iSLN. The iSLN also mediates the UAD reflex, where topical anesthesia can inhibit reflex GG activity and mechanical activation of airway receptors can augment TP muscle activity. Thus, electrical activation of this sensory nerve and/or other target nerves in OSA patients prevents UA obstructions by compensating for both the nocturnal attenuation of this reflex and the diminished airway mechanoreceptor function due to inflammation or submucosal adipose tissue. This may result in a stimulation-evoked increase of both electromyogram (EMG) activity of the UA dilator muscles (e.g., GG and/or TP), and UA patency (i.e., decrease in the critical pressure (P_{crit}) for flow limitation or collapse).

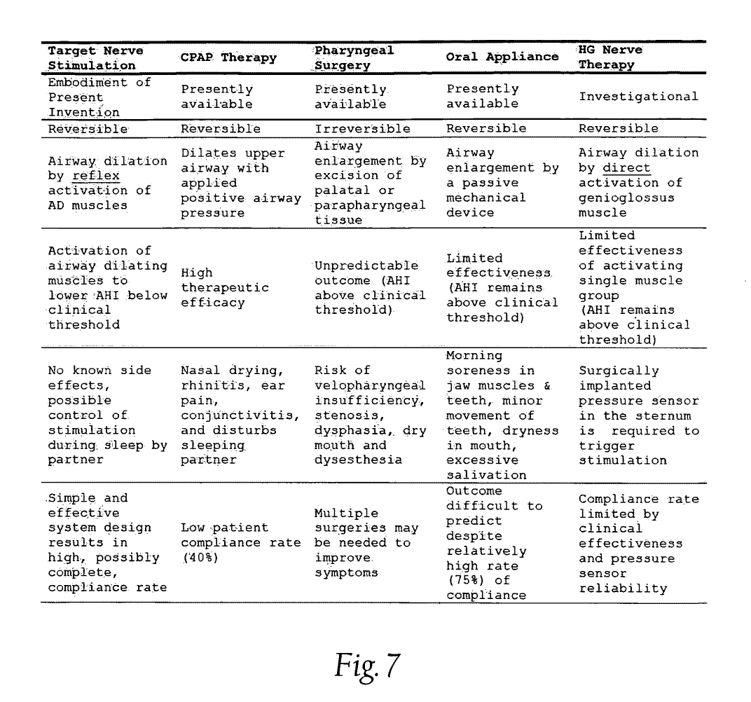

The potential advantages of electrically activating the UAD reflex by afferent nerve stimulation include (1) activation of UA dilator muscles (i.e., both GG and TP) that may contribute collectively to improve and/or maintain airway patency, (2) bilateral activation of UA dilator muscles in response to unilateral stimulation and (3) a simple system (e.g. implantable, external, percutaneous, and/or hybrid internal and external system) that does not necessarily require sensors to trigger stimulation. The advantage of co-activating multiple airway muscles may be important for maintaining airway patency as the therapeutic effect of the genioglossus muscle (i.e., HG nerve stimulation) can be significantly enhanced by activation of palatal muscles. Thus, electrical stimulation of the one or more target afferent nerves presents a novel approach to treating sleep-disordered breathing such as OSA, where the reflex activation of multiple UA dilating muscles can maximize the therapeutic potential of target nerve stimulation. The relative advantages and disadvantages associated with OSA therapy are summarized in the table shown in FIG. 7.

The cause of apneic events (i.e., insufficient airway dilator muscle activity) may be a disorder of the sensory receptor endings and/or a pathology of the central nervous system. Some or all of the AD reflex circuitry remains intact in sleep-disordered breathing (e.g. OSA) patients and the robust activation of the genioglossus muscle during HG nerve therapy precludes any myopathic causes of OSA. Thus, activation of the sensory fibers (e.g., target nerves, such as the internal branch of the superior laryngeal nerve (iSLN)) mediating the UAD reflex has been discovered to be a strong candidate for restoring airway patency.

Systems and methods according to the present invention provide a clinically effective therapy for sleep-disordered breathing such as OSA. The therapy may increase airway patency by activating an airway dilator (AD) reflex, such as an upper AD (UAD) reflex via electrical stimulation of one or more targeted afferent nerves, such as the internal branch of the superior laryngeal nerve (iSLN). The system may utilize an electrode placed and/or implanted in therapeutically effective proximity to the target nerve and connected to an electrical pulse generator, such as an implantable pulse generator (IPG) placed subcutaneously, preferably in the upper torso, as schematically depicted in FIG. 1. An example of an acceptable IPG to be used is disclosed in U.S. patent application Ser. No. 11/517,054, which is incorporated herein by reference in its entirety. Alternatively, the system may utilize one or more electrodes placed and/or implanted in therapeutically effective proximity to a target nerve and powered (via one or more wires or wirelessly) by an external power source or electrical pulse generator, such as an external pulse generator (EPG). During sleep, non-triggered electrical stimulation may be delivered to the target nerve, which has been demonstrated to be perceived as a comfortable and well tolerated sensation in humans. The stimulation may be provided continuously, according to a predetermined schedule, or according to a variable stimulation regime.

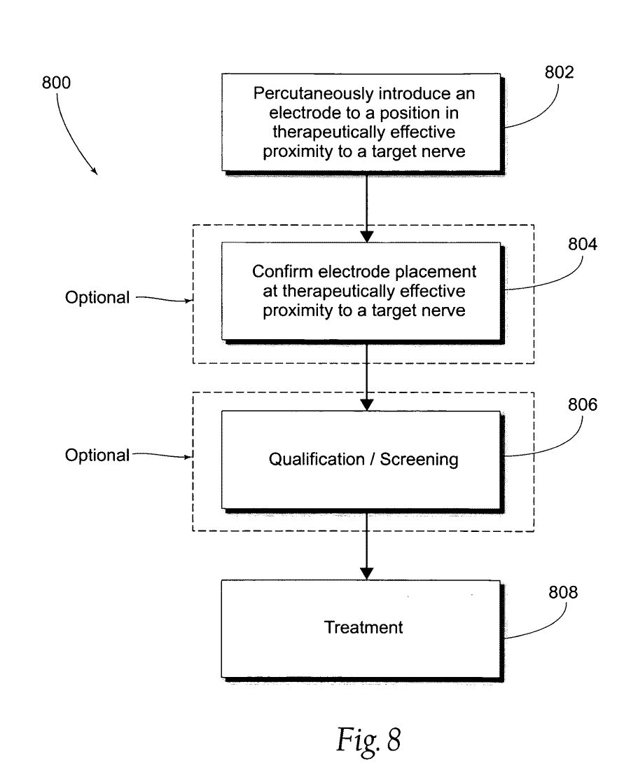

An embodiment 800 of a method according to the present invention is shown in FIG. 8. In a qualification or screening stage 802 of the method 800, an initial evaluation of stimulation-evoked reflex UAD muscle (e.g., genioglossus (GG) and/or tensor veli palatini (TP)) activity and/or increase in airway patency (measured as by favorable physiological parameters, such as a reduction in P_{crit}) may be conducted to determine whether a system or method according to the present invention may be effective treatment for a given patient. A preferred system to be used in the qualification stage 802 a percutaneous neurostimulation system, such as that disclosed in U.S. Patent Application 61/343,325, which is incorporated herein by reference in its entirety. For example, with the patient in a supine position and his or her upper airway and/or related structures anesthetized topically, subcutaneously, mucosally, or not at all, an electrode may be inserted (preferably after application of local anesthetic) percutaneously into the neck, to a depth of about 0.1 cm to about 8 cm, preferably about 0.2 cm to about 3 cm, and more preferably to a depth of about 0.2 cm to about 2 cm. The electrode insertion may be assisted by the use of diagnostic or metrological imaging, such as ultrasound, fluoroscopy, x-ray, computed tomography (CT), and/or magnetic resonance imaging (MRI). The electrode may be inserted between the cornu of the hyoid bone and the lateral aspect of the thyroid cartilage in proximity to the iSLN. The electrode may be a unipolar, single contact electrode, may be a unipolar multi-contact electrode, or it may be a multi-polar, multi-contact electrode. The electrode(s) may be connected to one or more pulse generators (e.g. IPG or EPG) via one or more wires or cables in the form of leads. Additionally or alternatively, one or more electrode(s) may be integrated with a pulse generator in a leadless configuration. Additionally or alternatively, the electrode(s) may be powered wirelessly by one or more internal and/or external power supplies and/or pulse generators. The electrode and/or lead may be inserted and/or anchored into adipose tissue, muscle tissue, or other tissue types in the neck in therapeutically effective proximity to the target nerve. The flexible anchors may keep the lead secure in the nearby adipose tissue, but are sufficiently flexible to allow removal without permanent damage to the tissue or the lead. An example of a preferred electrode arrangement to be inserted into adipose tissue is disclosed in U.S. patent application Ser. No. 11/290,736, which is incorporated herein by reference in its entirety. The same application discloses a suitable nerve cuff electrode. If the electrode is to be anchored in muscle tissue, a preferred intramuscular electrode may be used, such as that disclosed in U.S. Pat. No. 4,989,617, incorporated herein by reference in its entirety. The desired positioning of the electrode relative to a target nerve, such as the sensory branch of the SLN (iSLN), may be confirmed by 1) ultrasound imaging of the lead relative to anatomical landmarks, 2) the electrically evoked (e.g. by current pulses, such as those applied by an external stimulator) muscle activity, such as reflex muscle activity, which may be monitored visually, by palpation, and/or by electromyogram (EMG) activity of target muscles, such as the GG and/or TP (e.g. via paired wire electrodes, which may be paired or concentric, with latencies of multiple milliseconds, such as greater than 4 ms) muscles (similar to an example of which has been seen in preliminary animal studies, described below), and without concomitant muscle activity of the pharyngeal constrictor muscle (wire electrodes, latency <2-4 ms, FIG. 2), and/or 3) patient feedback, such as patient-reported sensation. Alternatively, concomitant reflex activation of both laryngeal and pharyngeal muscles may be desired in certain circumstances.

While some minor discomfort may be experienced by a patient, neither the insertion of the electrode nor the stimulation should cause significant pain. Indeed, the percutaneous insertion of a percutaneous electrode may even be accomplished without significant pain without using anesthetic.

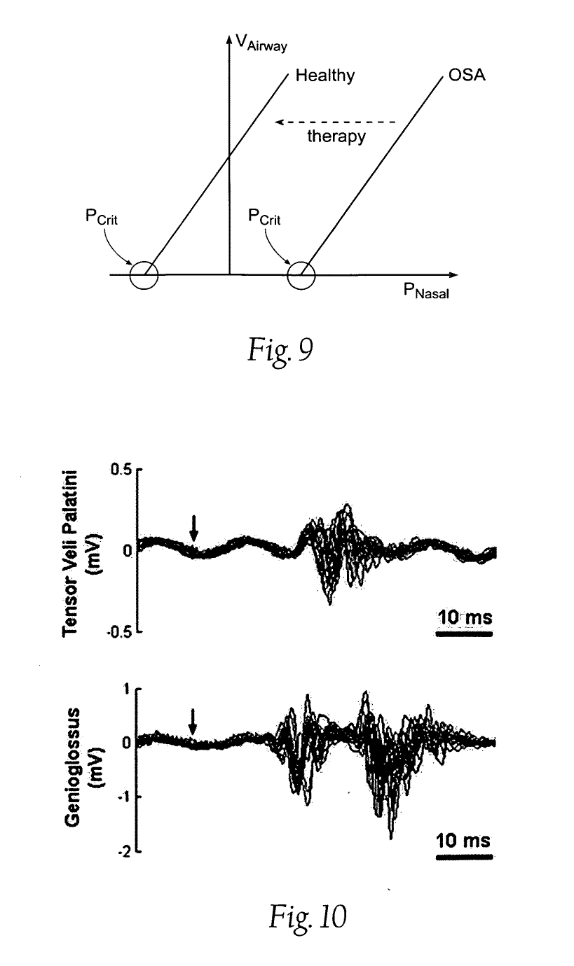

An optional verification step in the qualification stage of a treatment therapy according to the present invention may include fitting the patient with a nasal mask connected to an external pressure source, which may be used to adjust the airway pressure until flow limitation occurs. Patients in whom stimulation successfully activated airway muscles may proceed to a treatment stage 804 of the method 800. Success may be determined by a measure of a variety of physiological parameters. For instance, a successful or positive qualification or screening may be indicated by a reduction of a patient's P_{crit} below atmospheric pressure, as demonstrated by FIG. 9. FIG. 9 provides a pressure-flow diagram in which a left (negative) shift in P_{crit} represents a therapeutically effective outcome. A positive P_{crit} denotes upper airway collapse at positive nasal pressures. Another example of a successful or positive qualification or screening may be indicated by a reduction of a patient's apnea hypopnea index (AHI) below a predetermined level, such as below a clinical threshold (e.g. <5). While the qualification or screening stage 802 may be desirable, it is not absolutely necessary, and the following treatment stage 804 may be started without the qualification or screening stage 802, such as where a patient may present with indications suitable for treatment. Indications suitable for treatment may be based on collected, correlated data from prior, effective treatment stages involving other patients. The qualification stage 802, if implemented, may last from a few seconds to a month or more in duration. Preferably, the screening is performed as an in-home trial by the patient for a period of days, such as 1-3 days, 1-7 days, 1-14 days, and/or 1-30 days. Any of the implantations described herein, whether in connection with a percutaneous stimulation system or a completely implanted stimulation system, may be carried out in a relatively short (1-5 hour) outpatient procedure, a staged outpatient procedure occurring over several visits to a clinician, or a multi-day inpatient procedure.

To begin the treatment stage 804, the OSA patient may proceed to receive an implantable system, including the previously mentioned IPG. The external and/or percutaneous components of the percutaneous system, if used, may be discarded, the IPG may be placed in the body, and a standard IS-1 adapter cable may be tunneled subcutaneously from the IPG in the chest to the lead in the neck. The IPG is preferably placed subcutaneously in the upper torso. The IPG may be placed using conventional pocketing techniques. Additionally or alternatively, the IPG may be sutured in place, such as by using sutures coupled to the IPG header or other material attached to the IPG. Additionally or alternatively, the IPG may be coupled to a material that promotes or encourages in-growth of surrounding tissue. The IPG may be coated with such material, the material may be adhered to the IPG case, or a portion of the IPG case or housing may be made of such material. Based on clinical experience, this procedure may be an out-patient surgical procedure, preferably lasting less than three hours and more preferably lasting less than one hour, with a follow-up examination to test and determine optimum stimulation parameters. Patients may be monitored at various intervals such as a regular or irregular number of days, such as 1 to 7 days, a regular or irregular number of weeks, such as 1 to 4 weeks, and/or at a regular or irregular number of months, such as 1, 3, 6, and/or 12 months after implantation to assess therapeutic efficacy and safety.

The systems and methods according to the present invention may allow otolaryngologists to place and secure the electrode near the target nerve easily and reliably. The head, neck, and chest are areas that otolaryngologists are comfortable and familiar with, and unlike prior lines of treatment, embodiments of the present invention may be unobtrusive during sleep and may have a screening phase to indicate which patients may benefit from target nerve stimulation. Thus, this therapy is expected to be well received by otolaryngologists, sleep therapists, other clinicians, and their patients.

Electrical activation of a target afferent nerve, such as the internal branch of the superior laryngeal nerve (iSLN), which generates a reflexive response in a UAD muscle, offers a simple alternative to treating OSA by augmenting the upper airway dilator (UAD) reflex via afferent fibers that innervate airway mechanical receptors. This method is based on the realization and discovery of diminished airway mechanoreceptor function in OSA patients that result from chronic inflammatory-type damage to mucosal receptors from repeated snoring and also by sub-mucosal accumulation of adipose tissue. This loss in normal airway sensation is thought to play a critical role in rendering the upper airway vulnerable to flow-limitation and eventual collapse during sleep.

In animal studies, in 2 adult male cats with intact cortex and anesthetized with alpha-chloralose, the internal branch of the superior laryngeal nerve (iSLN) was exposed just proximal to laryngeal insertion, posterior to the thyroid cartilage. A bipolar nerve cuff electrode (0.8 cm length, contact area =1 mm×2 mm) was implanted on the iSLN and connected to a constant current stimulator. Pairs of stainless steel wires were inserted into the ipsilateral genioglossus (GG) and tensor veli palatini (TP) muscles to record stimulation-evoked electromyograms (EMG, gain=1000, filter=10 Hz to 1 kHz).

It has been demonstrated that electrical stimulation of the iSLN evokes reflex activity of the genioglossus (GG) and tensor veli palatini (TP) muscles.

Evoked EMG activity of the genioglossus (GG) and tensor veli palatini (TP) muscles was recorded in response to trains of electrical pulses (monophasic, pulse width=0.1 ms) applied to the iSLN at 1 Hz. The mean threshold for evoking a reflex EMG response was 0.18±0.17 mA (n=2). As shown in FIG. 10, the typical post-stimulus latency for both reflex GG and TP EMG signals was between 10 and 20 ms, suggesting a polysynaptic reflex circuit. FIG. 10 depicts a reflex EMG of the TP and GG muscles in response to a train of current pulses having a pulse width of about 0.1 ms and an amplitude of about 0.2 mA applied at a frequency of 1 Hz. Each plot shows 20 superimposed EMG traces with the stimulus pulse applied as at time indicated by the arrows. The stimulation method (i.e., bipolar) and the sufficient distance between the stimulating and recording electrodes minimized the stimulus artifact (arrows at t=0 s). A long-latency (30 ms) EMG response of the GG muscle (R2) indicated a secondary iSLN-to-GG reflex pathway. These results demonstrate that iSLN stimulation evokes reflex activation of both UA dilator muscles even under alpha-chloralose anesthesia.

It has also been demonstrated that the upper airway dilator (UAD) reflex persists during continuous stimulation of the iSLN.

The EMG activity evoked in both the GG and TP muscles during continuous electrical stimulation of the iSLN (amplitude=0.2 mA, frequency 1 Hz, duration=20 minutes) did not exhibit habituation of the UAD reflex, as shown in FIG. 11. FIG. 11 depicts reflex muscle activity of the tensor veli palatini (TP) and genioglossus (GG) muscles evoked by continuous stimulation of a target nerve, in this case the iSLN. The electrical stimulation was applied for a duration of about 20 minutes having a pulse width of about 0.1 ms, an amplitude of about 0.2 mA, and a frequency of about 1 Hz. The start of stimulation is indicated by the arrow. Upon termination of the iSLN stimulation train (not shown), reflex EMG activity of the GG and TP muscles completely stopped, indicating there was no significant prolonged (carry over) effect of the UAD reflex at 1 Hz stimulation. The TP EMG showed a burst of activity during the initial 20 seconds of stimulation before reaching a plateau and remaining consistently elevated above baseline for the duration of stimulation. Both the short (R1) and long latency (R2) components of the GG were elicited throughout the duration of nerve stimulation, and the amplitude of the evoked GG EMG activity was consistent during stimulation. Thus, prolonged stimulation of the iSLN evokes and sustains reflex contraction of both sets of the UAD muscles (GG and TP) for the duration of stimulation.

### Experimentation

A recent study in feline models has demonstrated the effectiveness of systems and methods according to the present invention. The study involved a total of 5 adult male cats. Each animal was sedated with xylazine (2.0 mg/kg, SQ) and anesthetized with ketamine HCl (15-25 mg/kg, IM). This was later switched to IV infusion of alpha-chloralose (15 mg/kg) via the cephalic vein. A tracheotomy was performed, where the caudal cut end was intubated and used to maintain normal respiration (end-tidal CO2 of 3-4%). Body temperature was maintained at 37° C.-39° C. with a heating pad, and IV fluids administered (˜15 cc/kg/hr) with 0.9% saline mixed with 8.4 mg/cc sodium bicarbonate and 5% dextrose.

1. Surgery and Instrumentation

A mid-cervical, longitudinal incision lateral to the thyroid cartilage exposed the internal branch of the superior laryngeal nerve (SLN) and the recurrent laryngeal nerve (RLN), which were implanted with bipolar and tripolar nerve cuff electrodes (inter-electrode distance=0.5 cm), respectively. The iSLN was electrically stimulated by a constant current stimulator (Pulsar 6 bp, FHC, Inc). Pairs of insulated stainless steel wires were inserted into the genioglossus (GG), and tensor veli palatine (TP) muscles. Both the electroneurogram (ENG) and electromyogram (EMG) were amplified (ENG=20,000, EMG=1000), filtered (ENG=100-3 kHz, EMG=10 Hz-2000 Hz), and digitally stored (sample rate=20 kHz). A subcutaneous needle inserted in the lateral thorax was used as a recording reference. In one cat, a second tracheal tube was placed in the rostral trachea, with the tip positioned caudal to the glottis, and connected in series with a pneumotachometer (PNT8411A, Hans Rudolph, Inc) and vacuum source.

2. Physiological Response to SLN Stimulation

In three cats, both the evoked ENG and EMG were measured in response to trains of monophasic, cathodic stimulus pulses delivered to the iSLN (pulse width=100 us, duration=20 s, frequency=1 Hz). The electrically evoked responses were recorded as both the stimulation amplitude (threshold up to 1 mA) and frequency (1, 5, 10, 20, and 40 Hz) were systematically varied. In one cat, stimulation evoked changes in UA patency were investigated. This involved measuring both the rate of airflow and the hypopharyngeal pressure (Php) during epochs (duration=1 to 2 seconds) of negative pressure applied by the vacuum source. These negative pressure pulses were applied throughout the experiment both without stimulation (i.e., baseline) and during 30-second trains of SLN stimulation (I=threshold to 1 mA, f=2, 10, 20 and 50 Hz).

3. Data Analysis

The electrically ENG and EMG responses were quantified by computing the rectified average of the signal within a specific time window, between 5 and 60 ms following the stimulus pulse. This window was reduced for higher stimulation frequencies (=20 Hz). For each set of stimulation parameter (i.e., amplitude+frequency), these responses were averaged over all pulses within the duration of the 20-second stimulus train. Changes in UA patency were quantified by the critical pressure at which flow limitation occurred in the cat. This was defined by determining the Php at which the first derivative (i.e., slope) of the measure airflow reaches an asymptote. Unless stated otherwise, all sets of data are represented as mean±standard deviation.

### Results of Experimentation

The physiological responses evoked by electrical stimulation of the SLN were recorded in 5 cats. The loss of upper airway reflex following complete transection of the nerve proximal to the stimulating electrode confirmed that (1) SLN afferents mediated this reflex and (2) adjacent vagus nerve afferents were not concomitantly activated (i.e., spillover) by SLN stimulation.

1. Reflex Activation of Upper Airway Muscles
a. Stimulation Threshold for Evoking Upper Airway Reflex

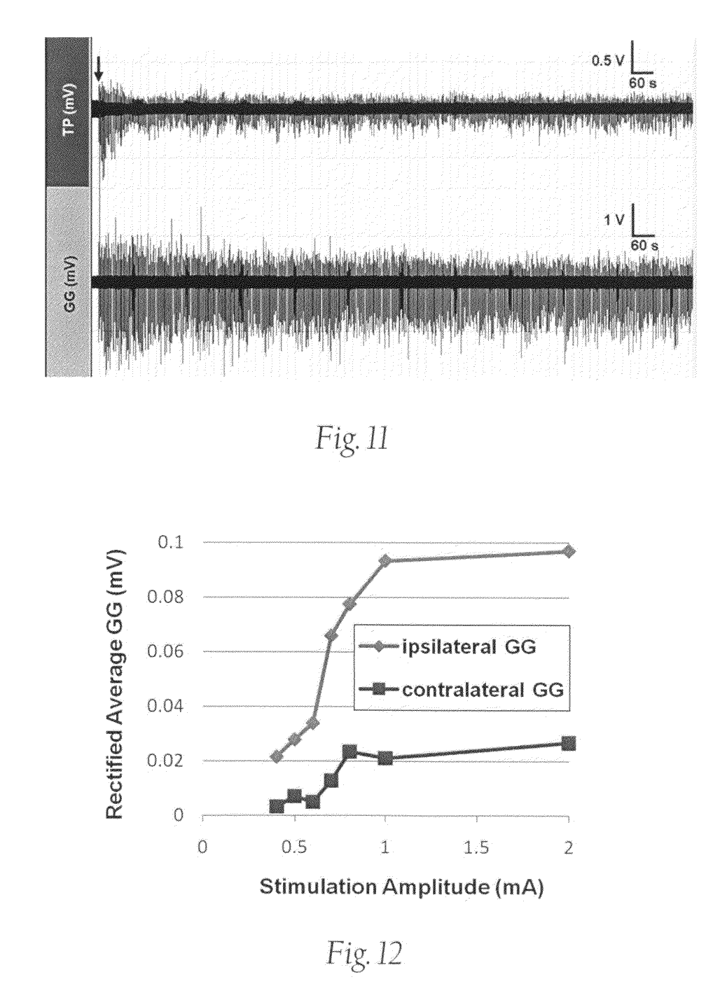

Electrical stimulation of the SLN evoked a reflex neural and/or muscular response in all 5 experiments. This was measured as EMG activity of the genioglossus (GG, 5 of 5 cats), and tensor palatini (TP, 2 of 3 cats) muscles (FIG. 12), and also as the ENG recorded from the recurrent laryngeal nerve (RLN, 2 of 2 cats). The mean stimulation amplitude required to evoke GG, TP and RLN responses were 0.16±0.19 mA (range=0.04 to 0.5 mA), 0.2±0.0 mA (range=0.2 to 0.2 mA), and 0.05±0.01 mA (range=0.04 to 0.05 mA), respectively. The mean ratio between the amplitude required for maximum GG response and that for threshold activity was 2.9±0.9 (n=5 cats).
b. Differential Latency of Upper Airway Reflex Pathways

Electrical stimulation of the SLN evoked reflex neural and muscular responses that exhibited notably different post-stimulus latencies between the neuromuscular structures supporting pharyngeal and laryngeal function. Reflex activity of the GG and TP muscles were measured at 19.1±8.3 ms (n=5) and 22.9±3.3 ms (n=2), respectively. In contrast, the reflex compound nerve action potential of the RLN measured just proximal to laryngeal innervation was 7.4±0.9 ms (n=2). A late reflex component of the GG (2 of 5 cats) and RLN (1 of 2 cats) was also observed in a smaller subset of animals: 30.9 ±2.5 ms and 53.9 ms, respectively.
c. Bilateral Activation of Upper Airway Dilator Reflex

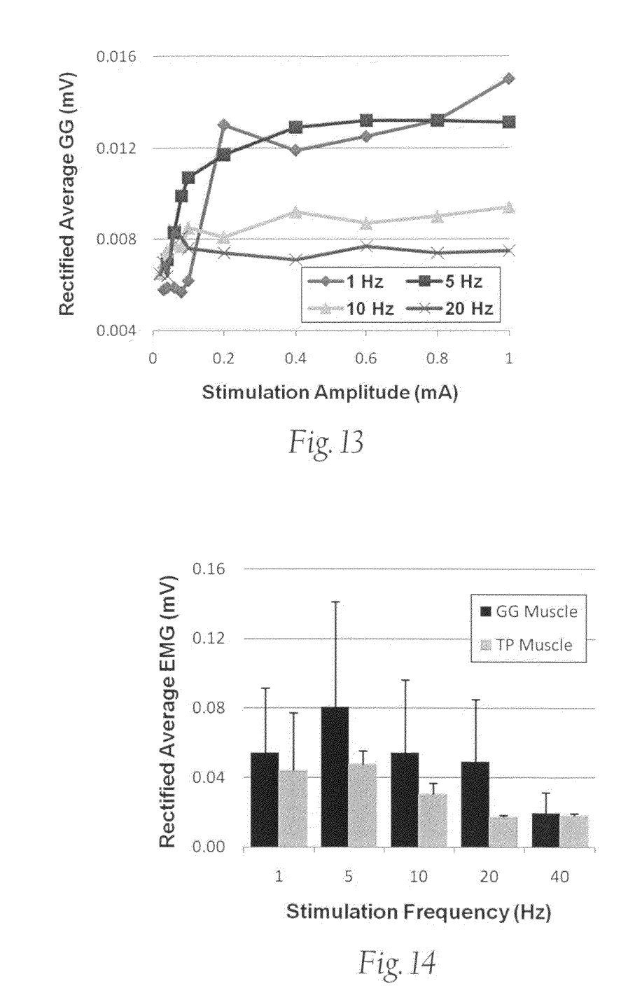

In one experiment (FIG. 13), measurement of both ipsi- and contralateral GG muscle demonstrated bilateral reflex activation of this airway dilator muscle. As shown in FIG. 12, the rectified average EMG of the ipsilateral GG muscle exhibited both a lower activation threshold (0.4 mA vs. 0.7 mA) and a larger maximum EMG response (0.097 mV vs. 0.027 mV) than that for the contralateral GG. Furthermore, compared to the maximum ipsilateral GG response evoked by direct hypoglossal nerve stimulation (rectified average EMG=0.197 mV), the reflex activity of the ipsi- and contralateral GG muscles evoked by SLN stimulation reached 49% and 14% of the physiological maximum, respectively.
d. Amplitude- and Frequency-Dependent Activation of Reflex Upper Airway Muscles

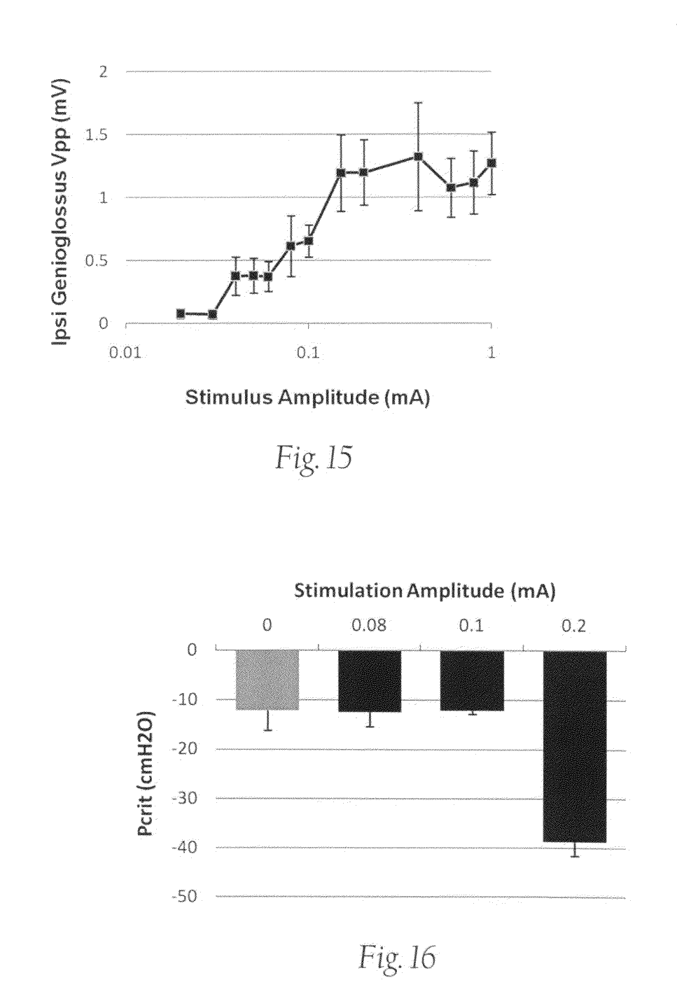

Electrical stimulation of the SLN (FIG. 14, n=1 cat) with 20-pulse trains of current pulses demonstrated an amplitude dependent recruitment of reflex GG muscle activity, where maximum response was achieved at approximately 0.2 mA. This pattern of muscle recruitment was consistent across all stimulation frequencies. In 3 separate cats (FIG. 15), electrical stimulation of the SLN evoked a maximum reflex response in both the GG and TP muscles at 5 Hz. At or above 10 Hz, there was an overall reduction in the evoked reflex activity; whereas individual EMG responses subsequent to the first pulse were significantly attenuated at 40 Hz.

2. Electrical Stimulation of the SLN Improves Upper Airway Patency
a. Electrical Recruitment of Reflex GG Muscle Activity

Electrical stimulation of the SLN using trains of 10 pulses applied at 1 Hz showed an increase in the evoked reflex GG response as the amplitude was increased (FIG. 16). The recruitment curve obtained in this experiment exhibited a marked difference in the stimulation thresholds for A and B type afferents of the SLN: 0.04 mA and 0.15 mA, respectively. As shown in the previous section, maximum recruitment of reflex GG activity is obtained at about 0.15 mA.
b. Amplitude-Dependent Change in Upper Airway Patency

Application of a vacuum source to the caudal end of the isolated upper airway resulted in a linear decrease in hypopharyngeal pressure (Php) with a corresponding rise in the rate of airflow through the upper airway. In all trials, the elastic properties of the airway mucosa resulted in flow limitation (FL) that was characterized by an asymptotic change in the airflow rate as the Php continued to decrease. The Php at which this occurred was defined as the critical pressure (P_{crit}). In this experiment, the baseline P_{crit} (n=15 trials) was −12.1±4.1 cmH2O.

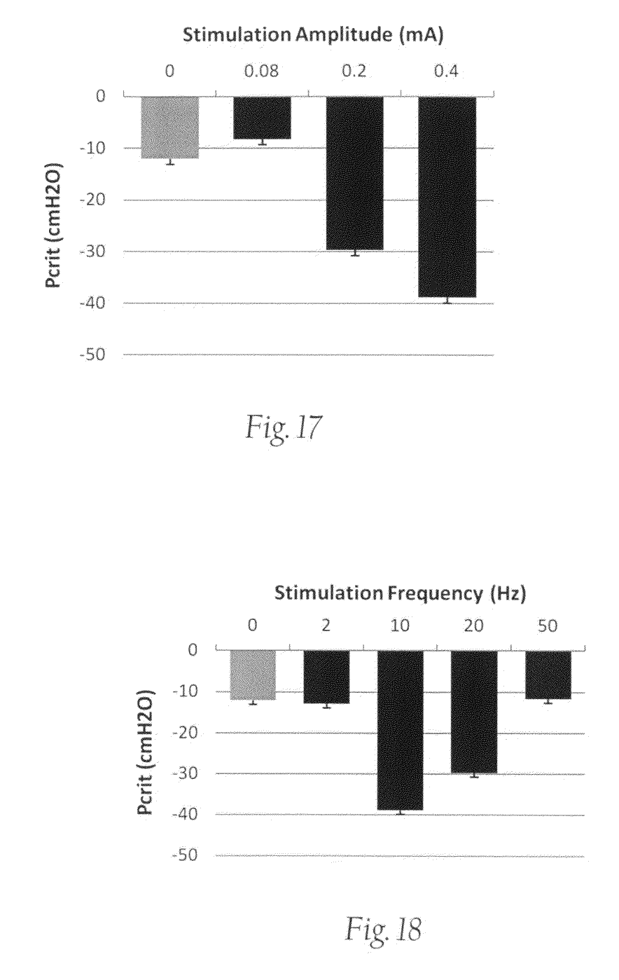

The negative pressure trials were repeated at least 2 times during continuous epochs of electrical stimulation of the SLN (duration=1-2 minutes). With the stimulation frequency set at 10 Hz, a 222% decrease in P_{crit} (−38.8±2.8 cmH2O) was observed at 0.2 mA (FIG. 16), which corresponded to the stimulation amplitude at which reflex GG activity reached maximum levels for SLN stimulation (FIG. 15). A similar pattern of response was observed during SLN stimulation at 20 Hz, where the measured P_{crit} exhibited 146% and 222% decreases at 0.2 mA and 0.4 mA, respectively (FIG. 17).
c. Frequency-Dependent Change in Upper Airway Patency

The effects of stimulation frequency on change in upper airway stability were also investigated in this cat. The stimulus amplitude was set above the threshold for maximum reflex GG activation (I=0.2 mA) and frequency was varied (f=2, 10, 20, and 50 Hz). As shown in FIG. 18, significant decreases in P_{crit} were observed during electrical SLN stimulation within the frequency range of 10-20 Hz. Electrical nerve stimulation outside this frequency window did not evoke any changes in P_{crit}.
d. Changes in Upper Airway Patency Correlate with Reflex Gg Activity

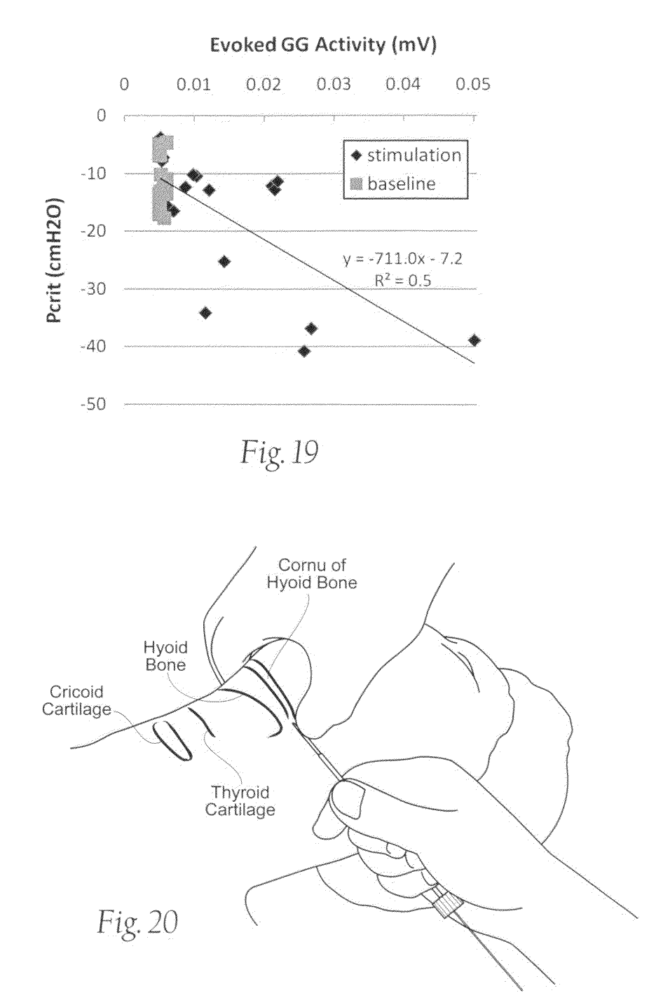

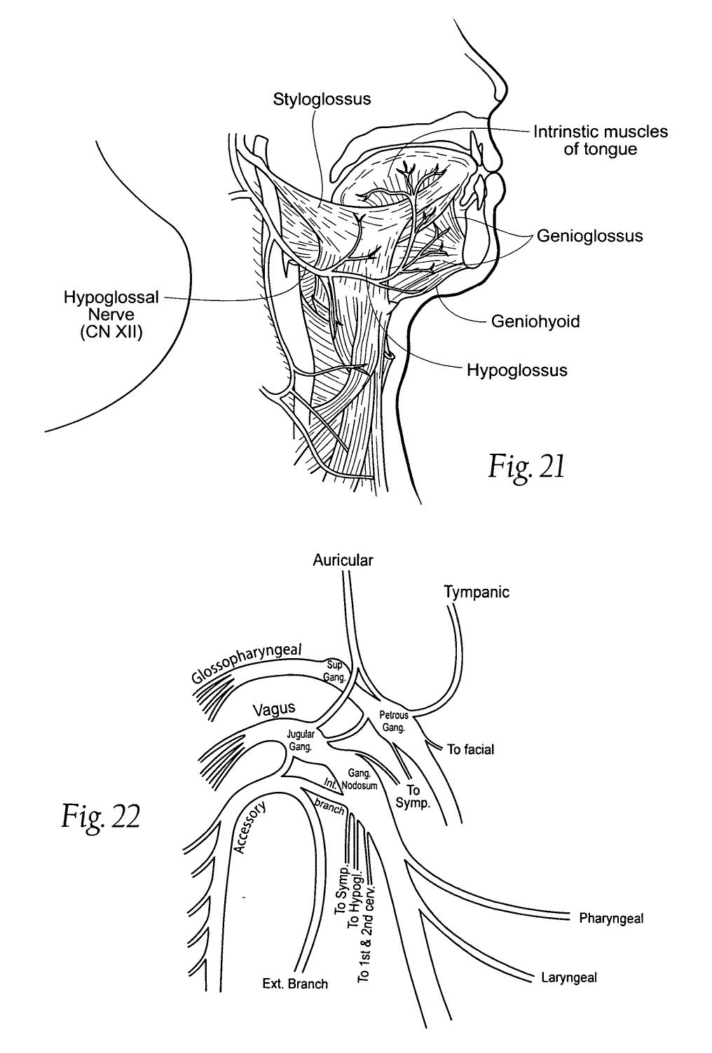

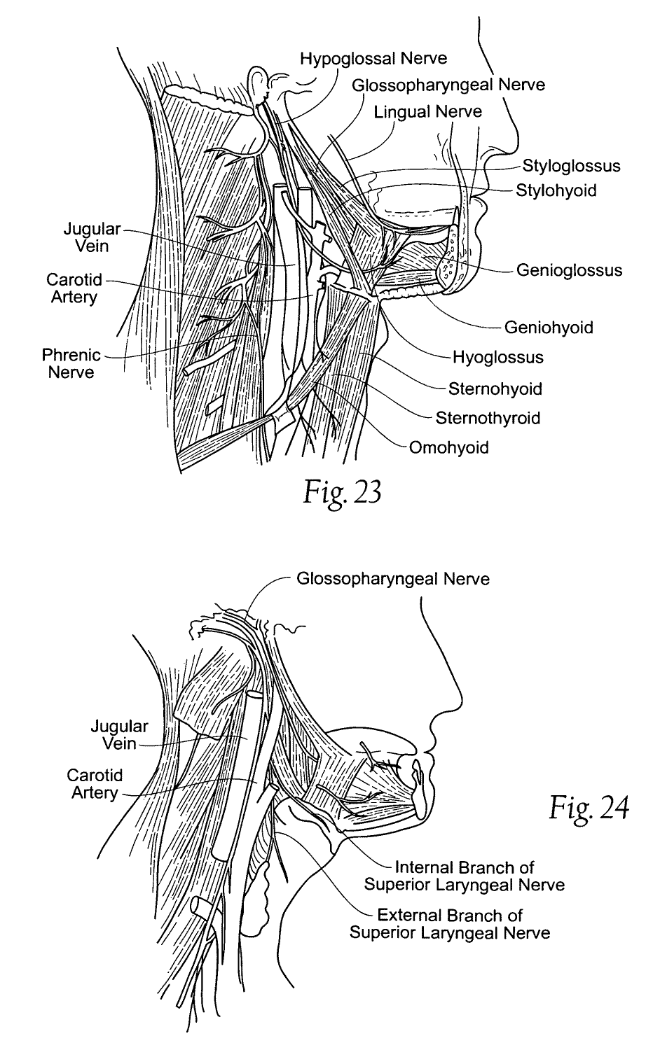

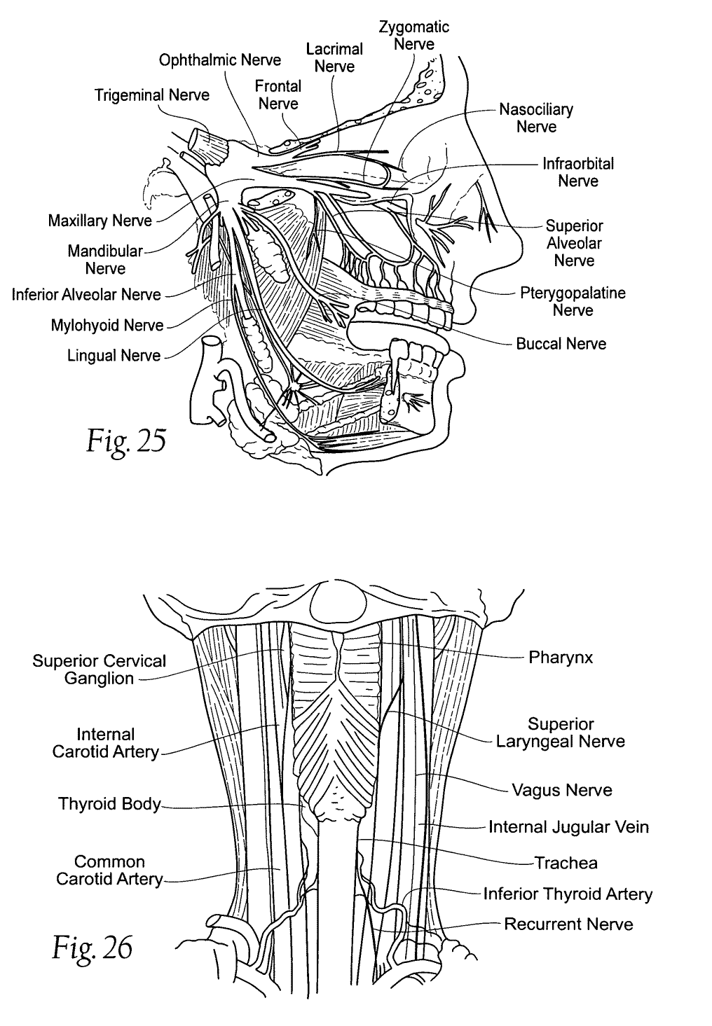

The measured changes in P_{crit} can be influenced by various factors such as the stimulation evoked activation level of the GG muscle and the rate of change in hypopharyngeal pressure. Analysis of the P_{crit} measured in relation to the electrically evoked GG muscle activity showed a strong positive relationship (FIG. 19, coefficient²=0.5): greater reflex GG activity correlated to lower P_{crit}. In contrast, the relationship between the measured P_{crit} and rate of change in the Php showed that this second variable (range=−4.6 cmH2O/s to −17.6 cmH2O/s) exerted a notable but weaker influence on P_{crit} (FIG. 19, coefficient²=0.2).

The results of this experimentation demonstrate that electrical stimulation of the superior laryngeal nerve (SLN) evokes robust reflex activity of the genioglossus (GG) and tensor palatini (TP) muscles in alpha-chloralose anesthetized cats. Furthermore, there was also a marked decrease in the critical airway pressure for flow limitation (P_{crit}) that was associated with SLN stimulation in one experiment. These findings indicate that electrical stimulation of airway afferents can maintain upper airway patency via reflex neural mechanisms (e.g., negative pressure reflex).

As demonstrated, the stimulation amplitude may play an important role in evoking the reflex activity of the upper airway dilator (GG and TP) and laryngeal (RLN electroneurogram) muscles. Consequently, the observed improvements in upper airway patentcy through muscle activation were positively correlated with the stimulus amplitude. Overall, the stimulation amplitude required to achieve threshold activation of these reflex pathways ranged from about 0.04 to about 0.15 mA, with one experiment requiring about 0.5 mA. More importantly, maximum reflex recruitment of these muscles were achieved at stimulus amplitudes that were less than 3 times the initial threshold. These values indicate that the upper airway excitatory reflex is afferently mediated by large diameter A-alpha and beta fibers that are easily activated by therapeutically effective electrical nerve stimulation. Interestingly, the stimulation evoked reflex responses (e.g., GG muscle) generated at amplitudes beyond 3 times the initial threshold were not affected by the larger stimulus inputs.

However, there was significant attenuation in the evoked reflex responses as the stimulation frequency was increased beyond 20 Hz. This refractoriness of the reflex was due to the time at which the reflex response terminated, typically about 60 ms post-stimulus. As a result, the reduced rectified average EMG and ENG reflex at frequencies above 20 Hz correlated with the diminished effects of SLN stimulation on reducing P_{crit}. Based on these preliminary findings, the optimum stimulation parameters for SLN therapy in OSA may be (1) stimulation amplitudes up to about three times the threshold for reflex muscle activity and (2) stimulation frequencies in the range of about 10 Hz to about 20 Hz. The refractoriness of the SLN mediated reflex pathways can also limit the duration of stimulation train. While low frequency stimulation (FIG. 11) can sustain reflex muscle activation over long periods of time (>10 minutes), frequencies (>10 Hz) that are more effective at improving upper airway patency may not exhibit this same pattern. Therefore, a human clinical implementation of SLN stimulation may involve (1) continuous stimulation throughout the night, (2) inspiratory effort triggered finite pulse trains (e.g., duration=1.5 s), (3) periodic stimulation, or (4) random stimulation.

The robust recording of SLN stimulation evoked reflex RLN activity indicates the putative role of the SLN as a protective mechanism during swallowing, such as preventing aspiration. This may negatively influence the intended therapeutic effects of SLN stimulation. The low stimulation amplitude required to activate this laryngeal activity suggests that large-diameter myelinated Aa-fibers afferently mediate this reflex pathway. Based on systems and methods according to the present invention, SLN therapy may activate the laryngeal musculature during stimulation. This is evidenced by the 2.5% and 31.7% increase in P_{crit} during SLN stimulation at 0.08 mA with pulses delivered at 10 Hz and 20 Hz respectively (FIGS. 16 and 17). However, as shown at higher stimulation amplitudes and at both frequencies, concomitant activation of reflex RLN and GG/TP muscles does not cause hypopharyngeal airway occlusion, but rather appears to promote airway patency. Thus, stimulation evoked reflex RLN activity may actually promote laryngeal stiffness by increasing the tension of the vocal cords (e.g., posterior cricoarytenoid muscle) and thus prevent passive glottis closure due to the negative pressures generated by inspiratory airflow.

In the animal model, the combined effects of a supine position and the application of a negative pressure pulses to the isolated pharynx may cause complete collapse of the upper airway. This could prevent measurement of upper airway flow rate, pharyngeal pressure and P_{crit}. As a solution, the tongue may be sutured or affixed to the lower lip to prevent prolapse during negative airway pressures, and the mouth may be sealed with suture and silicone epoxy.

The feline model exhibits an UAD reflex that is mediated by sensory fibers of the iSLN, similar to the mediation that occurs in humans. The presence of this reflex has been demonstrated by both direct (augmented genioglossus activity evoked by electrical stimulation of the iSLN) and indirect (loss of genioglossus activity by bilateral transection of iSLN) methods in felines. Finally, the feline iSLN exhibits a sensory innervation pattern that is remarkably similar to humans: dense fiber distribution within the oro- and laryngopharyngeal walls and the epiglottis. Overall, the cat nervous system has been the animal model of choice for the large majority of systems level neural studies because of its similarities to the human system. The somatic motor system and its reflexes bear a strong resemblance to what is known of human motor control.

### Surgical Procedure

Representative lead insertion techniques will now be described to place an electrically conductive surface in a desired location (e.g. in muscle tissue, adipose tissue, or other tissue) in therapeutically effective electrical proximity to a target nerve. It is to be understood that a therapeutically effective electrical proximity includes positioning the electrically conductive surface in direct contact with a target nerve, or spaced therefrom, but in any event able to be stimulated by electrical stimulation waveforms conducted through the electrically conductive surface.

Instructions for use can direct use of system and method for the placement of a lead in muscle in electrical proximity to the target nerve(s), including general outcome specification or guidance, or specific step-by-step instructions, including, e.g., the steps indicated herein, such as one or more of the steps included in the Surgical Procedure section, below. The instructions for use may include instructions for placing a lead for the activation of the targeted nerve of passage in a system for the relief of sleep apnea, for example. The instructions for use may also include instructions for recording stimulus parameters, including intensity associated with a first sensation of stimulation, a first noticeable muscle contraction, and a maximum tolerable contraction at multiple locations, which can be used to aid in determining desired stimulation parameters for optimal stimulation.

The instructions can, of course vary. The instructions may be physically present in one or more kits holding the lead but can also be supplied separately. The instructions can be embodied in separate instruction manuals, or in video or audio tapes, compact discs (CDs), and/or digital video discs (DVDs). The instructions for use can also or alternatively be available over an electronic communications network, such as through an internet web page.

To determine the optimal placement for the lead, test stimulation may be delivered through needle electrodes, and muscle responses may be observed. Needle electrodes may be used because they can be easily repositioned until the optimal location to deliver stimulation is determined.

At least one conductive lead and/or at least one electrode contact may be anchored in tissue (e.g. muscle, adipose, or other tissue) at a therapeutically effective electrical proximity to a target nerve. Such anchoring may result in the electrode contact being in physical contact with muscle tissue, nerve tissue, and/or adipose tissue. The electrode contact and/or lead may be inserted via an introducer in conventional fashion, which may be similar in size and shape to a hypodermic needle. The introducer may be any size. In a preferred embodiment, the introducer may range in size from 17 gauge to 26 gauge. Prior to inserting the introducer, the insertion site may be ,cleaned with a disinfectant (e.g. Betadine, 2% Chlorhexidine/80% alcohol, 10% povidone-iodine, or similar agent). One or more local anesthetics may be administered topically, mucosally, submucosally, and/or subcutaneously to the area in which the electrode and/or introducer will be inserted. To simplify the procedure, the insertion may be performed without anesthetic.

The position of the electrodes may be checked by imaging techniques, such as ultrasound, fluoroscopy, CT, MRI, and/or X-rays. Following placement of the lead(s), the portion of the leads which exit the skin may be secured to the skin using covering bandages and/or adhesives.

Electrical stimulation may be applied in an attempt to stimulate the target nerve(s) during and after placement of the electrode to determine whether stimulation of the target nerve can generate comfortable sensations and/or responses, such as airway dilation and/or target muscle contraction.

In a percutaneous and/or short-term therapy system the lead(s) and/or electrode contact(s) may be percutaneously placed near the target nerve and exit (if needed) at a skin puncture site. A trial or screening test may be conducted in a clinical setting (e.g. an office of a clinician, a laboratory, a procedure room, an operating room, etc.). During the trial, the lead is coupled to an external pulse generator and stimulation is provided by the generator through the lead, returning through a temporary percutaneous and/or surface return electrodes, to confirm the patient's response to stimulation by measuring various physiological parameters and/or patient feedback (e.g. comfortable sensations, airway dilation, increased and/or maintained airway patency, and/or target muscle contraction).

If the clinical screening test is successful, the patient may proceed to an overnight trial or a home-trial coupled to an external pulse generator and temporary percutaneous and/or surface return electrodes, to determine if the response (e.g. comfortable sensations, airway dilation, increased and/or maintained airway patency, and/or target muscle contraction) can be sustained in a sleeping and/or home environment. The trial period may range from minutes to hours to days to weeks to months. The preferred trial period may be between 1 and 21 days. If either the screening test or home trial is unsuccessful, the lead and/or electrode(s) may be readily removed.

However, if the screening test and/or home-trial are successful, the percutaneous system may be converted into a fully implanted system by replacing the external pulse generator with an implantable pulse generator (the housing of which may serve as a return electrode).

Alternatively, it may be preferred to use a percutaneous system(s) as a therapy without proceeding to a fully implantable system. It is also to be appreciated that a home-trial and/or a screening test is not a requirement for either the percutaneous system or a fully implanted system.

The duration of therapy for a percutaneous system may range from minutes to days to weeks to months to multiple years, but a preferred embodiment includes a duration ranging from 1 to 12 weeks.

Electrical stimulation may be applied between the active contact (located on a lead or the pulse generator) and return electrodes (located on a lead, the pulse generator, or elsewhere in a uni-polar or multi-polar mode). Regulated current is the preferred type of stimulation, but other type(s) of stimulation (e.g. non-regulated current such as voltage-regulated) may also be used. Multiple types of electrodes may be used, such as surface, percutaneous, and/or implantable electrodes. The surface electrodes may be a standard shape or they may be tailored if needed to fit the contour of the skin.

In a preferred embodiment of a percutaneous system, the surface electrode(s) may serve as the anode(s) (or return electrode(s)), but the surface electrode(s) may be used as the cathode(s) (active electrode(s)) if necessary. When serving as a return electrod(e), the location of the electrode(s) is not critical and may be positioned anywhere in the general vicinity, provided that the current path does not cross the heart. If a surface electrode(s) serves as an active electrode(s), it (they) may be positioned near the target stimulation area(s) (e.g. on the skin surface over the target nerve).

The electrode lead may be placed via multiple types of approaches. In one embodiment, the approach may be similar needle placement for electromyography (EMG) or nerve block.

For example if a target nerve includes nerves of the superior laryngeal nerve, such as the internal superior laryngeal nerve (iSLN) the approach can include a plurality of the following steps:

1) Place the patient in a comfortable and/or appropriate position (e.g. supine) with head tilted back and/or extended.

2) Prepare the lead insertion site with antiseptic and local subcutaneous anesthetic (e.g., 2% lidocaine).

3) Locate the site of skin puncture with appropriate landmarks, such as the hyoid bone, the cornu of the hyoid bone, cricoids cartilage, thyroid cartilage, thyrohyoid membrane, median thyrohyoid ligament, lateral thyrohyoid ligament, superior cornu of thyroid cartilage, superior laryngeal artery, median cricothyroid ligament, conus elasticus, cricothyroid muscle, cricothyroid joint, inferior cornu of thyroid cartilage, cricoid cartilage, and/or trachea.

It should be appreciated that the iSLN may branch from the superior laryngeal nerve (SLN) lateral to the greater cornu of the hyoid bone. The nerve may be located inferior (e.g. less than approximately 2-4 cm, less than approximately 1 cm, and/or less than approximately 5 mm) to the greater cornu of the hyoid bone, where it may cross the thyrohyoid membrane and may be located in the pyriform recess under the mucosa.

The cornu of the hyoid bone may be identified by palpation below the mandible. Identification of the landmark may be assisted by palpating along the upper border of the thyroid cartilage (from the thyroid notch) until reaching the cornu of the hyoid bone just superior to the posterior-lateral margin. The hyoid bone may be displaced with contralateral pressure to bring the iSLN and the ipsilateral cornu toward the clinician. This displacement may be performed with the clinician's non-dominant hand. This maneuver may allow the pulse of the carotid artery to be palpated below, sometimes deeply below, the finger of the clinician.

4) Insert a sterile electrode, set of electrodes, contacts, or lead (which may be preloaded in an introducer needle) at an angle based on landmarks used. The introducer may be inserted (e.g. in an anteroinferomedial direction) toward the lateral aspect of the greater cornu and toward the midline, slightly inferior to the lower border of the greater cornu. This insertion may or may not pierce the thyrohyoid membrane. An example of such insertion may be seen in FIG. 20.

Alternatively, a sterile electrode, set of electrodes, contacts, or lead (which may be preloaded in an introducer needle) may be inserted towards the pre-epiglottic space to access the target nerve(s), such as the iSLN. The pre-epiglottic space may be accessed lateral (e.g. less than approximately 2-4 cm) to the thyroid notch. The introducer may be inserted superoposteriorly toward the thyrohyoid membrane, which may or may not be pierced.

Alternatively, a sterile electrode, set of electrodes, contacts, or lead (which may be preloaded in an introducer needle) may be inserted superoanteromedially to the thyroid cornu.

5) Place a surface stimulation return electrode (such as a surface stimulation lead) in proximity of the area in which the percutaneous lead or electrode has been placed. Test stimulation will be applied to the lead, with the surface electrode providing a return path. The surface electrode may be placed adjacent to the lead. Its position is not critical to the therapy and it can be moved throughout the therapy to reduce the risk of skin irritation.

Alternatively, another type of return electrode may be used in place of a surface electrode. The return electrode may be located close to the active electrode(s) (e.g. cathode(s)) or some distance away from the active electrode(s).

Alternatively, a multi-polar style lead or electrode may be used, in which case the cathode and anode may be included on the same unit and may not need to be placed separately.

6) Couple the lead and return electrode to the external pulse generator. Set the desired stimulation parameters. Test stimulation may be delivered using a current-regulated pulse generator, for example. The external pulse generator may be programmed to generate electrical stimulation having ranges of amplitude, pulse duration, and frequency of 1-50 mA, 10-300 μs, 10-100 Hz, respectively, delivered for a desired stimulation time, such as about 0.1 seconds to about 0.2 seconds, and at a desired rate, such as about one to about 10 times per second, as a non-limiting example.

7) Advance the introducer slowly until the subject reports the first evoked sensation in the region innervated by the target nerve(s), or until desired physiological parameters are measured. Progressively reduce or increase the stimulus intensity and advance the introducer more slowly until the sensation can be evoked in the region innervated by the target nerve(s) at a predetermined stimulus amplitude (e.g., 1-10 mA). Stop the advancement of the introducer, and increase the stimulus amplitude in small increments (e.g., 0.1-0.5 mA) until the desired response is obtained. It is to be appreciated that any stimulation parameter, including pulse width and/or frequency, may be modulated in addition to or instead of amplitude.

It is to be appreciated that areas innervated by the superior laryngeal nerve (SLN) include the base of the tongue, the arytenoids, aryepiglottic fold, and the surface (e.g. the posterior surface) of the epiglottis. Thus, movement and/or patient sensation in any one or more of the areas innervated by the SLN may be used to guide selection of a stimulus regime (e.g. stimulus parameters, timing, etc) and/or placement of lead(s), introducer(s), and/or electrode contact(s).

8) Withdraw the introducer, leaving the percutaneous lead electrically conductive surface in therapeutically effective electrical proximity to the target nerve.

9) Cover the percutaneous exit site and external adjacent portion of the lead with a bandage. A bandage may also be used to secure the external portion of the lead (or an extension cable used to couple the lead to the external pulse generator) to the skin. It is expected the length of time to place the lead to be less than 10-20 minutes, although the process may be shorter or longer.

10) Vary the stimulus amplitude in small steps (e.g., 0.1-0.5 mA) to determine the thresholds at which stimulation evokes first sensation (T_{SEN}), muscle twitch (T_{MUS}) of the target muscle (innervated or not innervated by the target nerve), and maximum comfortable sensation (T_{MAX}). Query the subject at various stimulus amplitude to determine sensation level, and visually monitor muscle response. In addition to or instead of visually monitoring muscle responses, EMG may also be recorded. Threshold levels may be recorded. An initial map of the stimulation parameter space for evoking target muscle (e.g., GG and TP) EMG activity may be determined by applying trains of current pulses (intensity=threshold to 10×threshold (mA), frequency=1 Hz, 20 pulses, pulse width=0.1 ms), although other pulse parameter ranges may be used, such as about 1 Hz to about 150 Hz, individual to continuous pulses, and pulse widths about 1 μsec to about 10,000 μsec. The thresholds for generating direct and reflex target muscle activity may be defined by the respective nerve stimulated to elicit an EMG response.

11) It is possible that stimulation intensity may need to be increased slightly during the process due to causes such as habituation or the subject becoming accustomed to sensation, but the need for increased intensity is unlikely and usually only occurs after several days to weeks to months as the tissue encapsulates and the subject accommodates to stimulation. It is to be appreciated that the need for increased intensity could happen at any time, even years out, which would likely be due to either lead migration or habituation, but may also be due to reasons ranging from nerve damage to plasticity/reorganization in the central nervous system.

12) If the desired response cannot be evoked with the initial lead placement, redirect the introducer.

13) If sensations still cannot be evoked in a given subject, then the muscle twitch response of the muscle innervated or not innervated by the target nerve (e.g. muscle(s) in which reflex activity may be evoked by stimulation of the target nerve(s)) may be used to guide lead placement and then increase stimulus intensity until the desired response is obtained.

15) If desired, one or more additional leads or set of one or more electrode contacts (not shown) may be placed using a similar method to stimulate the nerves that are not activated by the first lead. For example, the lead(s) and/or contacts may be placed bilaterally to activate nerves on both the left and right side.

16) If stimulation is successful, i.e., if the screening test and/or home-trial are successful, the patient's percutaneous system may be converted into a fully implanted system by replacing the external pulse generator with an implantable pulse generator that is implanted in a convenient area. Success of the screening test and/or home-trial may be determined by achieving desired levels in measured physiological parameters, airway patency, airway muscle contractions, and/or patient or bed partner feedback. In one embodiment, the electrode lead used in the screening test and/or home-trial may be totally removed and discarded, and a new completely implantable lead may be tunneled subcutaneously and coupled to the implantable pulse generator for the treatment stage. In an alternative embodiment, a two part lead may be incorporated in the screening test and/or home-trial where an implantable part remains completely under the skin after implantation and is connected to a percutaneous part (i.e., extension) that can be discarded after removal. The implantable part may be of a predetermined length, adapted to extend to a desired implantation site of the IPG. The implantable part may then be tunneled towards and coupled to the implantable pulse generator, or a new sterile extension may be used to couple the lead to the implantable pulse generator.

If a target nerve includes nerves of the glossopharyngeal nerve the approach can include a plurality of the following steps:

1) Place the patient in a comfortable and/or appropriate position (e.g. supine or other position), preferably with head tilted back and/or extended.

2) Prepare the lead insertion site with antiseptic and local (e.g. subcutaneous or topical) anesthetic (e.g., 2% lidocaine or similar) if desired. In some patients, subcutaneous anesthetic may not be needed and/or may be intentionally avoided.

3) The target nerve can be approached intra-orally or extra-orally (e.g. peristyloid approach).

4) Extra-oral approach: a line may be drawn between the mastoid process and the angle of the mandible. The styloid process may be palpated (e.g. with deep pressure applied by the clinician) along this line just posterior to the jaw angle. The introducer can be positioned in the general direction of the styloid process but may be positioned slightly away and/or posterior from the styloid process (e.g. bony contact may be avoided).

Intra-Oral Approach: the mouth will be opened and the mucosa may be anesthetized. Insert a sterile electrode, set of electrodes, contacts, or lead (which may be preloaded in an introducer needle) below the mucosa at the base of the palatoglossal fold (or arch). The intra-oral approach may be preferable in situations where leadless, as opposed to leaded, electrical stimulators are utilized.

5) Place a surface stimulation return electrode in proximity of the area in which the percutaneous lead or electrode has been placed. Test stimulation will be applied to the lead, with the surface electrode providing a return path. The surface electrode may be placed adjacent to the lead. Its position is not critical to the therapy and it can be moved throughout the therapy to reduce the risk of skin irritation.

6) Couple the lead to the external pulse generator and to the return electrode. Set the desired stimulation parameters. Test stimulation may be delivered using a current-regulated pulse generator, for example. The external pulse generator may be programmed to 1-50 mA, 10-300 μs, 10-100 Hz, delivered for a desired stimulation time, such as about 0.1 seconds to about 0.2 seconds, and at a desired rate, such as about one to about 10 times per second, as a non-limiting example.

7) Advance the introducer slowly until the subject reports the first evoked sensation in the region innervated by the target nerve(s). Progressively reduce or increase the stimulus intensity and advance the introducer more slowly until the sensation can be evoked in the region innervated by the target nerve(s) at a predetermined stimulus amplitude (e.g., 1-10 mA). Stop the advancement of the introducer, and increase the stimulus amplitude in small increments (e.g., 0.1-0.5 mA) until the desired response is obtained. It is to be appreciated that pulse width may also be modulated in addition to or instead of amplitude. Utmost care should be taken during advancement of the introducer so as to avoid unintentional damage to other anatomical structures, such as blood vessels, especially arteries such as the carotid artery, and lymphatics.

It is to be appreciated that areas innervated by the glossopharyngeal nerve include parts of the tongue (e.g. the posterior portion), the soft palate, the oropharynx, and the surface (e.g. the pharyngeal surface) of the epiglottis. The target nerve(s), such as the glossopharyngeal nerve, may have multiple branches innervating multiple areas and structures, such as the tonsils (e.g. which may be innervated by the tonsillar branch), the pharynx and/or walls of the pharynx (e.g. which may be innervated by the pharyngeal branch), the epiglottis (e.g. such as the anterior surface, which may be innervated by the lingual branch) the vallecula, and the tongue (e.g. the posterior portion). Stimulation of the nerve may also mediate the gag reflex and/or changes in perception of the gag reflex, and or changes in perception in the nasotracheal, posterior pharynx, and/or related areas and/or responses may be used Thus, movement and/or patient sensation in any one or more of the areas innervated by the nerve may be used to guide selection of stimulus regime (e.g. stimulus parameters, timing, etc) and/or placement of lead(s), introducer(s), and/or electrode contact(s).

Branches of the target nerve may also be targeted for stimulation individually or in combination with or without stimulation of the entire target nerve and/or the other branches and/or nerve roots or related nervous structures.

If a target nerve includes nerves of the trigeminal nerve and/or branches of the trigeminal nerve such as but not limited to the nasociliary nerve(s) (and/or external nasal, internal nasal, short and long ciliary nerves and/or branches), pterygopalatine (and/or nasal branches, nasopalatine nerve(s), greater palatine nerve(s), lesser palatine nerve(s), and/or pharyngeal branch(es)), posterior superior alveolar nerve(s), infraorbital nerve(s) (e.g. middle superior alveolar, anteriori superior alveolar, inferior palpebral, lateral nasal, and/or superior labial branches), the approach can include:

1) Place the patient in a comfortable and/or appropriate position (e.g. supine) with head tilted back and/or extended.

2) Prepare the lead insertion site with antiseptic and local subcutaneous anesthetic (e.g., 2% lidocaine).

3) Locate the site of skin puncture with appropriate landmarks. The location of the mucobuccal fold may be identified above the maxillary first premolar. This location may serve as an insertion site. The infraorbital notch may be identified on the inferior orbital rim. The infraorbital foramen (which may be marked) may be identified in line with the second premolar, slightly inferior to the infraorbital notch. The foramen location may be noted while retracting the lip.

4) Insert a sterile electrode, set of electrodes, contacts, or lead (which may be preloaded in an introducer needle) at an angle based on landmarks used. The introducer may be inserted toward the infraorbital foramen above the first premolar at the approximate height of the mucobuccal fold. The maxillary bone may be avoided by orienting the introducer in line with the long axis of the first premolar. The introducer may be inserted but stopped short of making contact with bone.

5) Place a surface stimulation return electrode in proximity of the area in which the percutaneous lead has been placed. Test stimulation will be applied to the lead, with the surface electrode providing a return path. The surface electrode may be placed adjacent to the lead. Its position is not critical to the therapy and it can be moved throughout the therapy to reduce the risk of skin irritation.

6) Couple the lead to the external pulse generator and to the return electrode. Set the desired stimulation parameters. Test stimulation may be delivered using a current-regulated pulse generator, for example. The external pulse generator may be programmed to 1-50 mA, 10-300 μs, 10-100 Hz, and an on-off duty cycle of 0.25 sec., as a non-limiting example.

7) Advance the introducer slowly until the subject reports the first evoked sensation in the region innervated by the target nerve(s). Progressively reduce or increase the stimulus intensity and advance the introducer more slowly until the sensation can be evoked in the region innervated by the target nerve(s) at a predetermined stimulus amplitude (e.g., 1-10 mA). Stop the advancement of the introducer, and increase the stimulus amplitude in small increments (e.g., 0.1-0.5 mA) until the desired response is obtained. It is to be appreciated that pulse width may also be modulated in addition to or instead of amplitude.

Movement and/or patient sensation in any one or more of the areas innervated by the trigeminal nerve may be used to guide selection of stimulus regime (e.g. stimulus parameters, timing, etc) and/or placement of lead(s), introducer(s), and/or electrode contact(s).

In a representative embodiment, during lead placement, the stimulator is set to a frequency (e.g. 0.5-12 Hz (or 0.1-20 Hz, or 0.05-40 Hz)) low enough to evoke visible muscle twitches (i.e. non-fused muscle contraction) and/or muscle contraction(s) of the targeted muscle(s) innervated by the target nerve, but high enough that that the target nerve will be activated before the lead is advanced beyond the optimal position. It is to be appreciated that the muscle contraction(s) used for guidance during lead placement may be evoked directly (via efferent fiber activation) or indirectly (via afferent activation of a reflex that generates efferent activation).

The patient is not required to give verbal, written, or other type of feedback or indication of what they feel as the lead is being advanced towards the target nerve(s) if muscle contraction or imaging is used to guide lead placement, but patient feedback during lead advancement may improve lead placement in some patients. As non-limiting examples, those sensations reported by the patient may include first sensation (minimum stimulus intensity that evokes a sensation), level of comfort, maximum tolerable sensation, pain, qualities &/or descriptions of the sensations.

As an alternative to using muscle twitch(es) or contraction(s) as indicator(s) of lead placement (distance from the target nerve to electrode contact), patient sensation could instead be used to indicate lead location relative to the target nerve. Any combination of stimulus parameters that evoke sensation(s) may be used. Some stimulus parameters may evoke a more desirable response (e.g. more comfortable sensation, or a sensation that may be correlated with or specific to the specific target nerve fiber(s) within the target nerve. As an example, higher frequencies (e.g. =12 Hz, or =4 Hz, or =0.1 Hz) may evoke sensation(s) in the region(s) innervated by the target nerve(s) and though they may (or may not) also evoke muscle contraction(s), the muscle contraction(s) may not be noticeable (e.g. stimulus intensity may not be sufficient to evoke a contraction or a twitch from the present lead location or stimulus intensity may be sufficient to evoke contraction but the muscle contraction is fused (and no longer visually twitching), making it difficult to observe visually, unless EMG is used). To take advantage of both potential indicator responses (muscle twitch and patient sensation), higher frequencies may be applied intermittently (at lower frequencies), where the higher frequencies (e.g. 20-120 Hz, or 12-200 Hz) would normally caused fused muscle contraction if they were applied continuously but they are applied at an intermittent frequency (e.g. 0.5-4 Hz, or 0.1-11 Hz) that is low enough to allow the muscle to relax during the gaps between the bursts of stimulation, making it easier to visualize while still generating patient sensation at a higher frequency, allowing both muscle twitch and patient sensation to be used simultaneously as indicators of lead location relative to the target nerve.

While stimulation is being applied, the lead (non-limiting examples of the lead could include a single or multi-contact electrode that is designed for temporary (percutaneous) or long-term (implant) use or a needle electrode (used for in-office testing only)) may be advanced (e.g. slowly advanced) towards the target nerve until the desired indicator response (e.g. muscle twitch, muscle contraction, patient sensation, and/or some combination) is obtained. The intensity may then be decreased (e.g. gradually decreased) as the lead is advanced (e.g. advanced slowly) closer to the target nerve until the desired indicator response(s) may be obtained at smaller intensity(ies) within the target range (e.g. 0.1-1.0 mA (or 0.09-39 mA, or 0.009-199 mA), 100-300 us (or 40-1000 us, or 1- 10,000 us)) at some distance (e.g. X2 mm, where X2<X1, and (as a non-limiting example) X1 may be multiple times larger than X2, such as X1=2*X2, or X1=5*X2, or X1=20*X2) from the target nerve. If specific response(s) (e.g. desired response(s) and/or undesired response(s)) can be obtained at a range of intensities that are too low, then the lead may be located in a non-optimal location (e.g. too close to the target nerve(s)). Non-limiting examples of ranges of intensities that may be considered too low include those that are a fraction (e.g. <⅔, or <⅕, or < 1/10) of the intensities that obtained the desired response(s) at X1.

After a completed implantation of the IPG and implantable lead with electrode, the IPG may be programmed to generate electrical stimulation according to a variety of regimes. Preferably, controllers are provided to communicate transcutaneously with the IPG so as to enable programming and IPG control. A preferred clinician controller is disclosed in U.S. Pat. No. 7,761,167, which is incorporated herein by reference in its entirety. A preferred patient controller is disclosed in U.S. patent application Ser. No. 11/712,379, which is incorporated herein by reference in its entirety.

Control of the stimulator and stimulation parameters may be provided by one or more external controllers. In the case of an external stimulator, the controller may be integrated with the external stimulator. The implanted pulse generator external controller (i.e., clinical programmer) may be a remote unit that uses RF (Radio Frequency) wireless telemetry communications (rather than an inductively coupled telemetry) to control the implanted pulse generator. The external or implantable pulse generator may use passive charge recovery to generate the stimulation waveform, regulated voltage (e.g., 10 mV to 20 V), and/or regulated current (e.g., about 10 μA to about 50 mA). Passive charge recovery is one method of generating a biphasic, charge-balanced pulse as desired for tissue stimulation without severe side effects due to a DC component of the current.

The neurostimulation pulse may by monophasic, biphasic, and/or multi-phasic. In the case of the biphasic or multi-phasic pulse, the pulse may be symmetrical or asymmetrical. Its shape may be rectangular or exponential or a combination of rectangular and exponential waveforms. The pulse width of each phase may range between e.g., about 0.1 μsec. to about 1.0 sec., as non-limiting examples. The preferred neurostimulation waveform is cathodic stimulation (though anodic will work), biphasic, and asymmetrical.

Pulses may be applied in continuous or intermittent trains (i.e., the stimulus frequency changes as a function of time). In the case of intermittent pulses, the on/off duty cycle of pulses may be symmetrical or asymmetrical, and the duty cycle may be regular and repeatable from one intermittent burst to the next or the duty cycle of each set of bursts may vary in a random (or pseudo random) fashion. Varying the stimulus frequency and/or duty cycle may assist in warding off habituation because of the stimulus modulation.

The stimulating frequency may range from e.g., about 1 Hz to about 300 Hz, and the frequency of stimulation may be constant or varying. In the case of applying stimulation with varying frequencies, the frequencies may vary in a consistent and repeatable pattern or in a random (or pseudo random) fashion or a combination of repeatable and random patterns.

In a representative embodiment, the stimulator is set to an intensity within a desired range (e.g. 1-2 mA (or 0.1-50 mA, or 0.01-300 mA), 100-300 us (or 40-1000 us, or 1-10,000 us)) sufficient to activate the targeted nerve at some distance (e.g. 1 mm) away (from the targeted nerve). If the stimulus intensity is too great, it may generate muscle twitch(es) or contraction(s) sufficient to disrupt correct placement of the lead. If stimulus intensity is too low, the lead may be advanced too close to the target nerve (beyond the optimal position), possibly leading to incorrect guidance, nerve damage, mechanically evoked sensation (e.g. pain and/or paresthesia) and/or muscle contraction (i.e. when the lead touches the nerve), inability to activate the target nerve fiber(s) without activating non-target nerve fiber(s), improper placement, and/or improper anchoring of the lead (e.g. the lead may be too close to the nerve and no longer able to anchor appropriately in the muscle tissue).

Stimulus intensities may need to be scaled to account for biological factors, including but not limited to patient body size, weight, mass, habitus, age, and/or neurological condition(s). As a non-limiting example, patients that are older, have a higher body-mass index (BMI), and/or neuropathy (e.g. due to diabetes) may need to have stimulus intensities scaled higher (or lower) accordingly. Additionally, if electrode shape, geometry, or surface area were to change, then the stimulus intensities may need to change appropriately. For example, if the intensities were calculated for a lead with an electrode surface area of approximately 20 mm², then they may need to be scaled down accordingly to be used with a lead with an electrode surface area of 0.2 mm² because a decrease in stimulating surface area may increase the current density, increasing the potential to activate excitable tissue (e.g., target and non-target nerve(s) and/or fiber(s)). Alternatively, if the intensities were calculated for a lead with an electrode surface area of approximately 0.2 mm², then the intensities may need to be scaled up accordingly to be used with a lead with an electrode surface area of 20 mm². Alternatively, stimulus intensities may need to be scaled to account for variations in electrode shape or geometry (between or among electrodes) to compensate for any resulting variations in current density. In a non-limiting example, the electrode contact surface area may be 0.1 mm² to about 20 mm², or 0.01 mm² to about 40 mm², or 0.0001 mm² to about 1000 mm². In a non-limiting example, the electrode contact configuration may include one or more of the following characteristics: cylindrical, conical, spherical, hemispherical, circular, triangular, trapezoidal, raised (or elevated), depressed (or recessed), flat, and/or borders and/or contours that are continuous, intermittent (or interrupted), and/or undulating.

As mentioned above, if the lead is too far away from the target nerve of passage, then stimulation may be unable to evoke the desired response at the desired stimulus intensity(ies). If the lead is too close to the target nerve, then stimulation may be unable to evoke the desired response(s) (e.g. airway dilation) at the desired stimulus intensity(ies) without evoking undesirable response(s) (e.g. unwanted and/or painful muscle contraction(s) and/or sensation(s)). In some cases, it may be difficult to locate the optimal lead placement (or distance from the target nerve and/or it may be desirable to increase the range stimulus intensities that evoke the desired response(s) without evoking the undesired response(s) so alternative stimulus waveforms and/or combinations of leads and/or electrode contacts may be used. A non-limiting example of alternative stimulus waveforms may include the use of a pre-pulse to increase the excitability of the target fiber(s) and/or decrease the excitability of the non-target fiber(s).

Those skilled in the art will recognize that, for simplicity and clarity, the full structure and operation of all devices and processes suitable for use with the present invention is not being depicted or described herein. Instead, only so much of an implantable pulse generator and supporting hardware as is unique to the present invention or necessary for an understanding of the present invention is depicted and described. The remainder of the construction and operation of the IPGs described herein may conform to any of the various current implementations and practices known in the art.

Preferably, a kit according to the present invention comprise two or more of the following: external pulse generator, percutaneous lead including electrode, percutaneous lead introducer, implantable lead including electrode, implantable lead tunneling device, implantable pulse generator, clinician controller, instructions for use, and patient controller. The instructions for use

Stimulation of one or more target nerves (i.e. sensory pathways mediating an airway dilator reflex) may provide robust activation of airway dilator muscles (genioglossus (GG) and tensor veli palatini (TP)).

Electrical stimulation of such target nerve(s) may provide and/or restore the afferent input necessary to maintain airway patency (e.g. by activating a dilator reflex, such as the UAD reflex). Stimulation of the target nerve(s) can be more effective than stimulation of efferent pathways (such as those in the hypoglossal (HG) nerve) because in addition to GG activation (which can be achieved by target nerve stimulation and HG stimulation), target nerve stimulation can also activate other muscles, such as the TP muscles, to stiffen the airway, reducing the pressure the surrounding tissue places on the upper airway (UA) and ultimately preventing collapse of the airway, e.g. the upper airway, during inspiration.

Prolonged stimulation of one or more target nerves may sustain consistent and sufficient reflex activity in target muscles, such as the GG and TP, for the duration of stimulation. Repeated and/or continuous stimulation may or may not introduce temporal dependence to responses (e.g., fatigue, adaptation). Fatigue may not change the muscle response significantly because previous results in a dog model demonstrate that stimulation-evoked contractions are repeatable for a period of several hours. The sustained EMG response of the GG and TP muscles to iSLN stimulation suggests habituation is unlikely, but if habituation is observed, other stimulation regimes, intensities, frequencies and shorter bursts, or pulse duration, (e.g., shorter, longer, or variable duty cycle) of stimulation may be used to augment the reflex and minimize long-term habituation, adaptation, and/or fatigue of the response. Bursts and gaps of stimulation may be effective in optimizing reflex responses, and stimulus parameters may be optimized to maintain the reflex response for extended durations of continuous or non-continuous stimulation in humans.

While systems and methods according to the present invention have been described with detailed reference to target nerve stimulation, other systems and methods are contemplated. For instance, if unilateral stimulation of a target nerve fails to reduce sufficiently the critical pharyngeal pressure (P_{crit}) at flow limitation, then bilateral stimulation may be conducted. This may overcome the possible limitation of bilateral activation of airway muscles in which the efferent drive to both the ipsi- and contralateral muscles may not be equal in magnitude. Alternatively or additionally, a target nerve may be stimulated in synchrony with the inspiratory phase of respiration to augment the phasic activity of the hypoglossal nerve during inspiration.

Reflex inhibition of desirable reflex, such as efferent phrenic nerve activity, may occur during stimulation and may undermine the therapeutic effects of electrically activating the desired reflex, e.g. an upper airway dilator (UAD) reflex. If desirable activity decreases during stimulation, alternative stimulus parameters (e.g. lower intensity or higher intensity and short duration or long duration bursts of intermittent stimulation (less than 100% duty cycle) may be effective in activating the target reflex without inhibiting desirable (e.g. efferent phrenic nerve) activity.

The foregoing is considered as illustrative only of the principles of the invention. Furthermore, since numerous modifications and changes may readily occur to those skilled in the art, it is not desired to limit the invention to the exact construction and operation shown and described. For example, while treatment of OSA has been described in detail, embodiments of systems and methods according to the present invention may be employed to treat any indication where airway patency is desired, including but not limited to general, as opposed to only obstructive, sleep apnea. While the preferred embodiment has been described, the details may be changed without departing from the invention, which is defined by the following claims.

We claim:

1. A system for maintaining airway patency, the system comprising:
an electrical pulse generator adapted to produce electrical stimulation having a pulse duration, a frequency and an amplitude;
an electrode operatively coupled to the pulse generator, the electrode positioned within a therapeutically effective range relative to an afferent nerve in an animal body;
wherein, in response to the electrical stimulation produced by the electrical pulse generator and delivered to the afferent nerve through the electrode, at least one upper airway dilator muscle in the animal body is activated.

2. A system according to claim 1, wherein the electrode is a cuff electrode.

3. A system according to claim 2, wherein the electrode is positionable in direct substantially circumjacent contact with the afferent nerve.

4. A system according to claim 3, wherein the afferent nerve comprises the superior laryngeal nerve.

5. A system according to claim 4, wherein the afferent nerve comprises the internal branch of the superior laryngeal nerve.

6. A system according to claim 1, wherein the afferent nerve comprises one or more of the superior laryngeal nerve, the glossopharyngeal nerve, and the trigeminal nerve.

7. A system according to claim 1, wherein the electrode is adapted to be anchored in muscle tissue.

8. A system according to claim 7, wherein the electrode comprises:
an electrical conductor wound around a hollow core with gaps between adjacent windings;
a flexible, insulating sheath surrounding and encasing the electrical conductor;
a portion of the electrical conductor being wrapped around a periphery of a terminal end of the sheath to form an electrical contact;
a shaft of thermoplastic material in the hollow core and conforming with gaps between windings of the electrical conductor; and,
an anchor adapted for anchoring the electrode to tissue within which it is implanted, the anchor being connected with the thermoplastic shaft.

9. A system according to claim 1, wherein the electrode is adapted to be anchored in adipose tissue.

10. A system according to claim 9, wherein the electrode comprises:
an elongated lead, including one or more electrical conductors;
one or more electrically conductive surfaces, each conductive surface being electrically coupled to at least one of the electrical conductors and adapted to apply electrical stimulation to the afferent nerve; and
at least two expandable anchoring structures deployable from the lead to engage the adipose tissue and resist dislodgement and/or migration of the electrically conductive surfaces.

11. A system according to claim 1, wherein the electrode is a bipolar electrode.

12. A system according to claim 1 wherein the at least one upper airway dilator muscle is selected from the group consisting of the genioglossus muscle and the tensor veli palatini muscle.

13. A system according to claim 12 wherein the at least one upper airway dilator muscle comprises both the genioglossus muscle and the tensor veli palatini muscle.

14. A system according to claim 1, wherein the electrical stimulation further has a duty cycle and a stimulus regime, the system further comprising:
a clinician controller adapted to be placed in electronic communication with the pulse generator to allow remote adjustment of at least one of the pulse duration, the duty cycle, the stimulus regime, the amplitude and the frequency.

15. A system according to claim 14 further comprising a patient controller adapted to be placed in electronic communication with the pulse generator to allow remote adjustment of at least one of the pulse duration, the duty cycle, the stimulus regime, the amplitude and the frequency.

16. A system according to claim 15, wherein the patient controller is adapted to adjust at least one of the pulse duration, the duty cycle, the stimulus regime, the amplitude and the frequency within a predetermined set of parameters.

17. A system according to claim 1, wherein the electrical stimulation further has a duty cycle and a stimulus regime, the system further comprising:
a patient controller adapted to be placed in electronic communication with the pulse generator to allow remote adjustment of at least one of the pulse duration, the duty cycle, the stimulus regime, the amplitude and the frequency.

18. A system according to claim 17, wherein the patient controller is adapted to adjust at least one of the pulse duration, the amplitude and the frequency within a predetermined set of parameters.

19. A method for maintaining patency of an upper airway in an animal body, the method comprising the steps of:
anchoring an electrode within a therapeutically effective range relative to an afferent nerve in an animal body;
electrically coupling the electrode to an electrical pulse generator adapted to produce electrical stimulation having a pulse duration, a frequency and an amplitude; and
delivering the electrical stimulation from the pulse generator to the afferent nerve through the electrode to reflexively activate at least one upper airway dilator muscle in the animal body.

20. A method according to claim 19, wherein the electrode is a cuff electrode.

21. A method according to claim 20, wherein the electrode is positioned in direct substantially circumjacent contact with the afferent nerve.

22. A method according to claim 21, wherein the afferent nerve comprises the superior laryngeal nerve.

23. A method according to claim 22, wherein the afferent nerve comprises the internal branch of the superior laryngeal nerve.

24. A method according to claim 19, wherein the afferent nerve comprises one or more of the superior laryngeal nerve, the glossopharyngeal nerve, and the trigeminal nerve.

25. A method according to claim 19, wherein the anchoring step comprises the step of anchoring the electrode in muscle tissue.

26. A method according to claim 25, wherein the electrode comprises:
an electrical conductor wound around a hollow core with gaps between adjacent windings;
a flexible, insulating sheath surrounding and encasing the electrical conductor;
a portion of the electrical conductor being wrapped around a periphery of a terminal end of the sheath to form an electrical contact;
a shaft of thermoplastic material in the hollow core and conforming with gaps between windings of the electrical conductor; and,
an anchor adapted for anchoring the electrode to tissue within which it is implanted, the anchor being connected with the thermoplastic shaft.

27. A method according to claim 19, wherein the anchoring step comprises the step of anchoring the electrode in adipose tissue.

28. A method according to claim 27, wherein the electrode comprises:
an elongated lead, including one or more electrical conductors;
one or more electrically conductive surfaces, each conductive surface being electrically coupled to at least one of the electrical conductors and adapted to apply electrical stimulation to the afferent nerve; and
at least two expandable anchoring structures deployable from the lead to engage the adipose tissue and resist dislodgement and/or migration of the electrically conductive surfaces.

29. A method according to claim 19, wherein the electrode is a bipolar electrode.

30. A method according to claim 19 wherein the at least one upper airway dilator muscle is selected from the group consisting of the genioglossus muscle and the tensor veli palatini muscle.

31. A method according to claim 30 wherein the at least one upper airway dilator muscle comprises both the genioglossus muscle and the tensor veli palatini muscle.

32. A method according to claim 19 wherein the electrical stimulation further has a duty cycle and a stimulus regime, the method further comprising the step of:
placing a clinician controller in electronic communication with the pulse generator to remotely adjust at least one of the pulse duration, the duty cycle, the stimulus regime, the amplitude and the frequency.

33. A method according to claim 32 further comprising the step of placing a patient controller in electronic communication with the pulse generator to remotely adjust at least one of the pulse duration, the duty cycle, the stimulus regime, the amplitude and the frequency.

34. A method according to claim 33, wherein the patient controller is adapted to adjust at least one of the pulse duration, the duty cycle, the stimulus regime, the amplitude and the frequency within a predetermined set of parameters.

35. A method according to claim 19 wherein the electrical stimulation further has a duty cycle and a stimulus regime, the method further comprising the step of:
placing a patient controller in electronic communication with the pulse generator to remotely adjust at least one of the pulse duration, the duty cycle, the stimulus regime, the amplitude and the frequency.

36. A method according to claim 35, wherein the patient controller is adapted to adjust at least one of the pulse duration, the amplitude and the frequency within a predetermined set of parameters.
